(19) United States
(12) Patent Application Publication
(10) Pub. No.: US 2017/0049809 A1
(43) Pub. Date: Feb. 23, 2017

(54) COMPOSITIONS AND METHODS TO MODULATE ANGIOGENESIS

(71) Applicant: Fred Hutchinson Cancer Research Center, Seattle, WA (US)

(72) Inventor: Csaba Szabo, Seattle, WA (US)

(21) Appl. No.: 15/082,512

(22) Filed: Mar. 28, 2016

Related U.S. Application Data
(63) Continuation of application No. 14/172,407, filed on Feb. 4, 2014, now abandoned.
Application No. 14/172,407 is a continuation of application No. 12/664,872, filed on May 7, 2010, now abandoned.
(60) Provisional application No. 60/944,444, filed on Jun. 15, 2007.

(51) Int. Cl. A61K 33/04; A61K 9/00; A61K 33/00

(52) U.S. Cl. CPC A61K 33/04; A61K 33/00; A61K 9/0019

(57) ABSTRACT

The present invention provides methods of stimulating angiogenesis and the growth or migration of cells associated with angiogenesis, by contacting animals, tissues, or cells with sulfide, alone or in combination with nitric oxide. These methods may be used for a variety or purposes, including promoting wound healings, increasing blood flow, and for the treatment and prevention of diseases and disorders associated with decreased blood flow, including ischemic or hypoxic injury.

### CROSS-REFERENCE TO RELATED APPLICATIONS

This application claims the benefit under 35 U.S.C. Å119(e) of U.S. Provisional Patent Application No. 60/944,444, filed Jun. 15, 2007; where this provisional application is incorporated herein by reference in its entirety.

### FIELD OF THE INVENTION

The present invention relates to compositions and methods for modulating angiogenesis. These compounds and methods may be used for the prevention and treatment of angiogenesis-associated conditions such as wound healing and coronary or vascular diseases and disorders.

### DESCRIPTION OF THE RELATED ART

Angiogenesis or “neovascularization” refers to the development of new blood vessels and the branching and growth of capillaries composed of endothelial cells (Ziche et al., Current Drug Targets, 2004:5; 485-493). In mammals, angiogenesis ensures proper development of mature organisms and plays a key rote in reproduction as it prepares the womb for egg implantation. Angiogenesis has an important role in the body's response to injury, in tumor growth, wound healing, and chronic inflammatory diseases (see: WO/2007/005670; Folkman et al., Science, 235:442-447 (1987)).

New blood vessel formation is required for the development of normal and pathological tissue. Angiogenesis aids in the healing of wounds and fractures, the vascularization of synthetic skin grafts, and enhancement of collateral circulation in the event of vascular occlusion or stenosis. Regulation of angiogenesis is a likely control point in the regulation of many disease states, as well as a therapeutic opportunity for growth of normal tissue and regulation of disease (see: U.S. Pat. No. 6,191,144).

Angiogenesis is a multi-step process controlled by the balance of pro- and anti-angiogenic factors. The latter stages of this process include the proliferation and organization of endothelial cells (EC) into tube-like structures. Growth factors such as fibroblast growth factor 2 (FGF2) and vascular endothelial growth factor (VEGF) promote endothelial cell growth and differentiation. Inhibition of angiogenesis can be achieved by inhibiting endothelial cell responses to stimulators of angiogenesis (e.g., VEGF or bFGF; Folkman, J. Annu. Rev. Med., 57:1-18 (2006).

Angiogenesis occurs as a response to injury, in wound healing, myocardial ischemia, coronary artery disease, angina and peripheral vascular diseases. Excessive angiogenesis may be harmful and is observed in cancer, tumor growth, inflammation, arthritis, rheumatoid arthritis, psoriasis and ocular diseases. Excessive angiogenesis may be inhibited as a therapeutic to treat tumors and disease (Folkman, J. Annu. Rev. Med., 57:1-18 (2006).

Therapeutically, induction of angiogenesis is beneficial to patients in many pathological disease states including myocardial ischemia and peripheral vascular disease. Gene therapy induction (Ziche et al., Curr Drug Targets, 5:485-493 (2004)) or administration of bone marrow cells after stimulation with cytokines (Ferrar N., and Kerbel, R. S. Nature, 438:967-74 (2005)) have been shown to induce angiogenesis.

Clearly, there is a need in the art for compositions and methods that modulate angiogenesis. An effective pharmacologic therapy to modulate angiogenesis would provide substantial benefit to the patient, thereby avoiding the challenges of using gene therapy or cytokines. The present invention provides sulfide compositions that modulate angiogenesis in a beneficial manner.

### BRIEF SUMMARY OF THE INVENTION

The present invention provides novel compositions and methods for promoting, enhancing or stimulating angiogenesis, e.g., in an animal or an animal tissue or organ.

In one embodiment, the present invention provides a method of stimulating angiogenesis in a biological matter, comprising administering to the biological matter an effective amount of sulfide. In particular embodiments, the biological matter is an animal, e.g., a mammal, or an animal tissue or organ. The tissue or organ may be present within the animal or removed from the animal.

In various embodiments of the methods of the present invention, the sulfide is administered in a stable liquid pharmaceutical composition comprising said sulfide and a pharmaceutically acceptable carrier, wherein the concentration, pH, and oxidation products of said sulfide remain within a range of acceptance criteria after storage of said liquid pharmaceutical composition.

In certain embodiments, the stable liquid pharmaceutical composition is prepared by dissolving one equivalent of hydrogen sulfide gas into one equivalent of sodium hydroxide solution, wherein said composition has a pH in the range of 6.5 to 8.5, wherein said composition has an osmolarity in the range of 250-330 mOsmol/L, wherein said composition has an oxygen content of less than or equal to 5 μM, and wherein said composition comprises oxidation products are the range of 0%-3.0% (w/v) after storage for three months.

In a related embodiment, the present invention provides a method of stimulating angiogenesis in a biological matter, comprising administering to the biological matter an effective amount of sulfide in combination with an effective amount of nitric oxide.

In particular embodiments, nitric oxide and sulfide are administered as gases. In other embodiments, nitric oxide and sulfide are administered as liquids. In related embodiments, nitric oxide is administered as a gas and sulfide is administered as a liquid. In other related embodiments, nitric oxide is administered as a liquid and said sulfide is administered as a gas. Nitric oxide and sulfide may be administered concurrently. In other embodiments, sulfide is administered prior to administration of nitric oxide, or nitric oxide is administered prior to administration of sulfide.

In one embodiment, the biological matter is a mammal. In particular embodiments, the biological matter is a mammalian tissue or organ.

In a further related embodiment, the present invention provides a method for promoting re-epithelialization of a denuded area of skin of an animal, e.g., after a burn, trauma, wound, injury, chemotherapy, skin reaction following drug treatment or disease process, comprising administering to the animal an effective amount of sulfide, alone or in combination with an effective amount of nitric oxide.

In yet another related embodiment, the present invention provides a method for promoting wound healing in a patient, comprising administering to a patient an effective amount of sulfide, alone or in combination with an effective amount of nitric oxide. In various embodiments, sulfide is administered locally or topically.

In another embodiment, the present invention includes a method for increasing blood flow to ischemic tissue in a biological material, the method comprising: administering to the biological matter an amount of sulfide effective to stimulate angiogenesis and increase blood flow to said ischemic tissue.

Another embodiment of the present invention provides a method for treating or preventing an injury or disease associated with decreased or insufficient blood flow in a patient, comprising administering to said patient an effective amount of sulfide, alone or in combination with an effective amount of nitric oxide. The decreased or insufficient blood flow may be transient or chronic. It may be decreased or insufficient cerebral blood flow. In certain embodiments, the insufficient blood flow is localized within said patient. In particular embodiments, said injury or disease is diabetic foot ulcers, peripheral vascular disease, a coronary injury or disease, e.g., congestive heart failure, myocardial ischemia, coronary artery disease, or angina, or an ocular disease.

In a further related embodiment, the present invention provides a method of increasing, promoting, or stimulating growth, proliferation, or migration of a cell associated with angiogenesis, comprising contacting said cell with an effective amount of sulfur, alone or in combination with an effective amount of nitric oxide,

### BRIEF DESCRIPTION OF SEVERAL VIEWS OF THE DRAWING(S)

FIG. 1A is a graph showing the effect of increasing concentrations of a liquid formulation of sulfide (NaHS) on the chorioallantoic membrane (CAM). NaHS or vehicle (control) was applied onto 1 cm² of the CAM and incubated for 48 hours at 37° C. CAMS were fixed and excised from the eggs. The total length of the vessel network was measured using image analysis software. The graph depicts vessel growth (% of control) upon exposure to the indicated concentrations of NaHS. Results are expressed as means ±S.E.M.; p<0.05 versus the control.

FIG. 1B depicts representative photographs showing the CAM vascular network following treatment with vehicle (control; top panel) or with a liquid formulation of sulfide (NaHS; bottom panel).

FIG. 2A is a graph that compares Human Umbilical Vein Endothelial Cells (HUVEC) tube formation in Matrigel®-coated wells in 96-well plates in the presence of a liquid formulation of sulfide (60 μM NaHS) or vehicle (control) and incubated for 6 hours at 37+ C. The length of the tube network was measured in the total well area. Results are expressed as means ±S.E.M.; p<0.05 versus the control.

FIG. 2B depicts representative photomicrographs showing the formation of tube-like structures on Matrigel® after control (top panel) or 60 μM NaHS (bottom panel) treatment.

FIG. 3 is a graph that shows increasing proliferation rates of HUVEC cells with increasing concentrations of a liquid formulation of sulfide (6 μM, 60 μM, and 600 μM NaHS) assessed as a percentage of baseline measurement. The experiments were performed in duplicate at passage two, using 4-6 wells each time.

FIG. 4 is a graph that shows improved re-epithelialization in the presence of a liquid formulation of sulfide (NaHS) in a model of wound healing. Rats received a 30% total body surface area dorsal full-thickness scald burn under deep anesthesia. Starting at 48 hours post bum, the animals received daily subcutaneous injections at four equally spaced sites in the transition zone between burn eschar and healthy tissue. Planimetric measurement of the wound surface and re-epithelialization as well as the ratio of wound contraction were performed. Results are expressed as means ±S.E.M.; n=5; *p<0.05 versus the control.

FIG. 5A is a graph that shows a liquid pharmaceutical sulfide (NaHS) stimulates migration of endothelial cells. HUVEC were serum starved overnight and then trypsinized, placed in transwells, and allowed to migrate for 4 hours in the presence of a liquid formulation of sulfide (6 μM or 60 μM NaHS) or vehicle (control) at 37° C. Non-migrated cells at the top of the transwell filter were removed with a cotton swab. The migrated cells were fixed in Carson's solution for 30 minutes at room temperature and then stained in toluidine blue for 20 minutes at room temperature. Migrated cells were scored in 8 random fields and the fold-change was determined compared to the number of control wells. Results are expressed as means ±S.E.M.; n=5; *p<0.05 versus the control.

FIG. 5B depicts representative photomicrographs of the transwell membrane showing cell migration in vehicle (control; top panel) or liquid sulfide (NaHS; IK-1001) treatment (bottom panels).

FIG. 6 is a diagram depicting the pro-angiogenic and re-epithelialization effects of a liquid formulation of sulfide on tube-like formation, migration, proliferation and wound healing.

### DETAILED DESCRIPTION OF THE INVENTION

As used in the specification and appended claims, unless specified to the contrary, the following terms have the meaning indicated:

As used herein, the term “angiogenesis,” indicates the growth or formation of blood vessels. Angiogenesis includes the growth of new blood vessels from pre-existing vessels, as well as vasculogenesis, which refers to spontaneous blood-vessel formation, and intussusception, which refers to new blood vessel formation by splitting off existing ones. Angiogenesis encompasses “neovascularization”, “regeneration of blood vessels,” “generation of new blood vessels”, “revascularization,” and “increased collateral circulation.”

The terms “angiogenesis agent” and “angiogenic agent” refers to any compound or substance that stimulates, accelerates, promotes, or increases angiogenesis, whether alone or in combination with another substance.

The terms “anti-angiogenesis agent” and “anti-angiogenic agent” refer to any compound or substance that inhibits, prevents, or reduces angiogenesis, whether alone or in combination with another substance.

An “angiogenesis associated condition” includes any process, disease, disorder, or condition that is dependent upon or associated with angiogenesis. This term includes diseases, disorders, and conditions resulting from or associated with insufficient or reduced angiogenesis, as well as diseases, disorders, and conditions resulting from or associated with too much, unwanted, or increased angiogenesis. The term includes conditions that involve cancer, diabetes, ocular disorders and wound healing, as well as those that do not. An “angiogenesis dependent condition” is any disease, disorder, or condition that requires angiogenesis.” Angiogenesis dependent or angiogenesis associated conditions can be related to (e.g., arise from) unwanted angiogenesis, as well as with wanted or desired (e.g., beneficial) angiogenesis.

The term “re-epithelialization” refers to restoration of epithelium over a denuded area of skin. The term includes restoration of epithelium by natural growth, by grafting, i.e. plastic surgery, or during the process of wound healing. The process of re-epithelialization includes epithelial cell migration and proliferation leading to closure of the epithelia. Examples include re-epithelialization of skin after a burn, trauma, wound, injury, chemotherapy, skin reaction following drug treatment, or a disease process that results in injury or loss of epithelium of the skin.

The term “biological material” refers to any living biological material, including cells, tissues, organs, and/or organisms, and any combination thereof. It is contemplated that the methods of the present invention may be practiced on a part of an organism (such as cells, tissue, and/or one or more organs), whether that part remains within the organism or is removed from the organism, or on the whole organism. Moreover, it is contemplated in the context of cells and tissues, both homogenous and heterogeneous cell populations may be the subject of embodiments of the invention.

The term, “chronic” refers to a condition, symptom or disease which persists over a long period of time and/or is marked by frequent recurrence (e.g., chronic colitis). Chronic disease refers to a disease which is of long continuance, or progresses slowly, in distinction from an acute disease, which quickly terminates.

The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The terms “isolated” and “ex vivo” are used to describe such biological material. It is contemplated that the methods of the present invention may be practiced on in vivo and/or isolated biological material.

The cells treated according to the methods of the present invention may be eukaryotic or prokaryotic. In certain embodiments, the cells are eukaryotic. More particularly, in some embodiments, the cells are mammalian cells. Mammalian cells include, but are not limited to those from a human, monkey, mouse, rat, rabbit, hamster, goat, pig, dog, cat, ferret, cow, sheep, or horse.

Cells of the invention may be diploid but in some cases, the cells are haploid (sex cells). Additionally, cells may be polyploid, aneuploid, or anucleate. In particular embodiments, a cell is from a particular tissue or organ, such as one from the group consisting of: head, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord. In certain embodiments, cells are characterized as one of the following cell types: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotont or multipotent), unfertilized or fertilized oocyte, or sperm.

The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue. In certain embodiments, the tissue or organ is “isolated,” meaning that it is not located within an organism.

“Organism” includes but is not limited to, mammals, reptiles, amphibians, birds, fish, invertebrates, fungi, plants, protests, and prokaryotes. In particular embodiments, a mammal is a marsupial, a primate, or a rodent. In other embodiments, an organism is a human or a non-human animal. In specific embodiments, an organism is a mouse, rat, cat, dog, horse, cow, rabbit, sheep, fruit fly, frog, worm, or human.

“Optional” or “optionally” means that the subsequently described event of circumstances may or may not occur, and that the description includes instances where said event or circumstance occurs and instances in which it does not.

“Pharmaceutically acceptable carrier, diluent or excipient” includes without limitation any adjuvant, carrier, excipient, glidant sweetening agent, diluent, preservative, dye/colorant, flavor enhancer, surfactant, wetting agent, dispersing agent, suspending agent, stabilizer, isotonic agent, solvent, or emulslfier which has been approved by the United States Food and Drug Administration as being acceptable for use in humans or domestic animals.

“Administering” includes routes of administration which allow the compositions of the invention to perform their intended function, e.g., promoting or stimulating angiogenesis. A variety of routes of administration are possible including, but not necessarily limited to parenteral (e.g., intravenous, intra-arterial, intramuscular, subcutaneous injection), oral (e.g., dietary), topical, nasal, inhalation, rectal, or via slow releasing micro-carriers depending on the disease or condition to be treated.

“Effective amount” includes those amounts of an agent, e.g., an angiogenic compound, which allow it to perform its intended function, e.g., stimulating angiogenesis in angiogenesis-associated conditions as described herein. The effective amount will depend upon a number of factors, including biological activity, age, body weight, sex, general health, severity of the condition to be treated, as well as appropriate pharmacokinetic properties. It is understood that an effective amount of an agent, such as hydrogen sulfide, may be a different amount when the agent is used alone as compared to when it is used in combination with another agent such as, e.g., nitric oxide.

“Pharmaceutical composition” refers to a formulation of a compound and a medium generally accepted in the art for the delivery of the biologically active compound to mammals, e.g., humans. Such a medium includes all pharmaceutically acceptable carriers, diluents or excipients therefore.

“Prodrug” refers to a compound that may be converted under physiological conditions or by solvolysis to a biologically active compound of the present invention. Thus, the term “prodrug” refers to a metabolic precursor that is pharmaceutically acceptable. A prodrug may be inactive when administered to a subject in need thereof, but is converted in vivo to an active compound. Prodrugs are typically rapidly transformed in vivo to yield the active compound, for example, by hydrolysis in blood. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam)). A discussion of prodrugs is also provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

“Sulfide” refers to sulfur in its −2 valence state, either as H₂S or as a salt thereof (e.g., NaHS, Na₂S, etc.). Sulfide also refers to deuterium sulfide or ²HS. “H₂S” is generated by the spontaneous dissociation of the chalcogenide salt and H₂S donor, sodium hydrosulfide (NaHS), in aqueous solution according to the equations:

NaHS→Na++HS⁻

2HS⁻ [symbol] H₂S+S₂⁻

HS⁻+H+ [symbol] H₂S.

It was recently demonstrated that H₂S (hydrogen sulfide) gas, a potent inhibitor of oxygen consumption, can reduce metabolism and protect mice and rats from hypoxic injuries. It was shown that treatment with sulfide and other chalcogenides induces stasis and enhances survivability of biological matter and protects biological matter from hypoxic and ischemic injury (PCT Publication No. WO2005/041655). Although hydrogen sulfide gas has not been typically considered a medical gas, this unexpected result supports the use of sulfide for the treatment or prevention of a number of animal and human diseases, particularly hypoxia and ischemia-related diseases and injuries.

Sulfide has many physiological actions in mammals, including, but not limited to, vasodilatation, cytoprotection, metabolic depression (or stasis), and anti-inflammation. However, if has not previously been shown to play a role in angiogenesis. Sulfide has not yet been approved by the FDA for use in invasive medical intervention. However, when administered either parenterally or by inhalation/ventilation to mammals, sulfide reduces injury and enhances survivability in myocardial infarction, cardiac surgery, lethal hemorrhage, cerebral and hepatic ischemia, and lethal hypoxia. Sulfide may reduce injury or enhance survivability in similar or other human diseases or injuries.

While the embodiments of the present invention described herein are primarily directed to sulfur compounds, it is understood that in other embodiments, the present invention may be practiced using chalcogenides other than sulfur. In certain embodiments, the chalcogenide compound comprises sulfur, while in others it comprises selenium, tellurium, or polonium. In certain embodiments, a chalcogenide compound contains one or more exposed sulfide groups. In particular embodiments, it is contemplated that a chalcogenide compound contains 1, 2, 3, 4, 5, 6 or more exposed sulfide groups, or any range derivable therein. In particular embodiments, such a sulfide-containing compound is CS₂ (carbon disulfide).

In certain embodiments, the chalcogenide is a salt, preferably salts wherein the chalcogen is in a −2 oxidation state. Sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS); barium sulfide (BaS), and the like.

“Chalcogenide precursor” refers to compounds and agents that can yield a chalcogenide, e.g., hydrogen sulfide (H₂S), under certain conditions, such as upon exposure, or soon thereafter, to biological matter. Such precursors yield H₂S or another chalcogenide upon one or more enzymatic or chemical reactions. In certain embodiments, the chalcogenide precursor is dimethylsulfoxlde (DMSO), dimethylsulfide (DMS), methylmercaptan (CH₃SH), mercaptoethanol, thiocyanate, hydrogen cyanide, methanethiol (MeSH), or carbon disulfide (CS₂). In certain embodiments, the chalcogenide precursor is CS₂, MeSH, or DMS. Compounds on the order of the size of these molecules are particularly contemplated (that is, within about 50% of their molecular weights).

“Chalcogenide” or “chalcogenide compounds” refers to compounds containing a chalcogen element, i.e., those in Group 6 of the periodic table, but excluding oxides. These elements are sulfur (S), selenium (Se), tellurium (Te) and polonium (Po). Specific chalcogenides and salts thereof include, but are not limited to: H₂S, Na₂S, NaHS, K₂S, KHS, Rb₂S, CS₂S, (NH₄)₂S, (NH₄)HS, BeS, MgS, CaS, SrS, BaS, H₂Se, Na₂Se, NaHSe, K₂Se, KHSe, Rb₂Se, CS₂Se, (NH₄)₂Se, (NH₄)HSe, BeSe, MgSe, CaSe, SrSe, PoSe and BaSe.

It is well known in the art that sulfides are unstable compounds and produce oxidation products. As used herein, “sulfide oxidation product” refers to products that result from sulfide chemical transformation, including, e.g., sulfite, sulfate, thiosulfate, polysulfides, dithionate, polythionate, and elemental sulfur.

The invention disclosed herein is also meant to encompass metabolic products of the disclosed compounds and agents. Such products may result from, for example, the oxidation, reduction, hydrolysis, amidation, esteriftcation, and the like of the administered compound, primarily due to enzymatic processes. Accordingly, the invention includes compounds produced by a process comprising contacting a compound of this invention with a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Therapeutically effective amount” refers to that amount of a compound or agent that, when administered to a mammal, preferably a human, is sufficient to effect treatment, as defined below, of a disease or condition in the mammal, preferably a human. The amount of a compound or agent that constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure. It is also understood that a therapeutically effective amount of an agent, such as hydrogen sulfide, may be a different amount when the agent is used alone as compared to when it is used in combination with another agent such as, e.g., nitric oxide.

“Treating” or “treatment” as used herein covers the treatment of the disease or condition of interest, e.g., tissue injury, in a mammal, preferably a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition.

As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In certain embodiments, the present invention is directed to uses of stable liquid compositions comprising a chalcogenide, e.g., sulfide. For purposes of the present invention, the term “liquid” with regard to pharmaceutical compositions is intended to include the term “aqueous.”

In one aspect, the present invention relates to a stable, liquid pharmaceutical composition which comprises a chalcogenide or chalcogenide compound or salt or precursor thereof, wherein the concentration, pH, and oxidation products of said chalcogenide remain within a range of acceptance criteria (numerical limits, ranges, or other criteria for the tests described) after storage of said liquid pharmaceutical composition for a pre-specified time period.

As used herein “stable” refers to the concentration of the active chalcogenide composition, the pH of the chalcogenide composition and/or chalcogenide oxidation products remaining within a range of acceptance criteria.

“Acceptance criteria” refers to the set of criteria to which a drug substance or drug product should conform to be considered acceptable for its intended use. As used herein, acceptance criteria are a list of tests, references to analytical procedures, and appropriate measures, which are defined for a drug product that will be used in a mammal. For example, the acceptance criteria for a stable liquid pharmaceutical composition of chalcogenide refers to a set of predetermined ranges of drug substance, pH, and levels of oxidation products that are acceptable for pharmaceutical use for the specific drug composition based on stability testing. Acceptance criteria may be different for other formulations, include those for topical and cosmetic use. Acceptable standards are generally defined for each industry.

Various acceptance criteria include any value or range described herein that meets Good Manufacturing Practice Regulations promulgated by the US Food and Drug Administration. In certain embodiments, an acceptance criteria is a pH in the range of 7.4-9.0, 6.5 to 8.5, or 6.5 to 9.0 at a time point of 0, 1, 2, 3, or 4 months storage at 4° C., 25° C., or 40° C. In certain embodiments, an acceptance criteria is an osmolality in a range of 250-350 mOsm/kg or an osmolarity in the range of 250-330 mOsm/L at a time point of 0, 1, 2, 3, or 4 months storage at 4° C., 25° C., or 40° C. In certain embodiments, an acceptance criteria is a sulfide concentration of 5.0-6.0 mg/ml at a time point of 0, 1, 2, 3, or 4 months storage at 4° C., 25° C., or 40° C. In another embodiment, an acceptance criteria is a concentration of chalcogenide within the range of 0.1-100 mg/ml, 1-10 mg/ml, or 95-150 mM at a time point of 0, 1, 2, 3, or 4 months storage at 4° C., 25° C., or 40° C. In other embodiments, an acceptance criteria is sulfide present at least 80%, at least 85%, at least 90%, at least 95%, at least 98%, at least 97%, at least 98%, or at least 99% weight/volume of total sulfide and oxidation products thereof at a time point of 0, 1, 2, 3, or 4 months storage at 4° C., 25° C., or 40° C. In related embodiments, oxidation products are present at a concentration less than 10%, less than 5%, less than 4%, less than 3%, less than 2%, less than 1%, 0.5% or less of total sulfide and oxidation products at a time point of 0, 1, 2, 3, or 4 months storage at 4° C., 25° C., or 40° C.

### Methods of Stimulating Angiogenesis and Treating Angiogenesis Associated Diseases and Disorders

The present invention is based, in part, on the surprising discovery that sulfide stimulates angiogenesis. As described herein, contacting endothelial cells with increasing concentrations of sulfide, e.g., a liquid formulation of sulfide (NaHS), results in a dose-dependent increase in angiogenesis or neovascularization in a variety of different angiogenesis assays. Thus, the present invention establishes that sulfide is an agent that promotes or increases angiogenesis. Accordingly, the instant invention contemplates the pharmaceutical use of sulfide to promote or stimulate angiogenesis in vitro, ex vivo, and in vivo, e.g., in tissues and organisms, and provides compositions and methods for promoting and increasing angiogenesis in biological material, e.g., tissues, organs, organisms, and animals. In addition, these methods and compositions may be used to promote or increase growth or proliferation of cells, associated with angiogenesis, e.g., endothelial cells.

Given the relationship between angiogenesis and a variety of angiogenesis associated conditions, the present invention further includes compositions and methods for the treatment and prevention of angiogenesis associated conditions. In particular embodiments, the compositions and methods of the present invention are used to treat conditions associated with insufficient, reduced, or inadequate angiogenesis. In one embodiment, e.g., they are used to promote wound healing.

In one embodiment, the present invention includes methods of promoting, enhancing, or increasing angiogenesis in a biological matter, comprising contacting the biological matter with an effective amount of sulfide. In particular embodiments, the biological matter is mammalian, e.g., mammalian cells, tissue, organ or animal. In particular embodiments, the biological matter is an animal such as a mammal. In particular embodiments, the amount of angiogenesis is increased by at least 5%, at least 10%, at least 25%, at least 50%, at least 100%, at least 200%, at least 500% or at least 1000% as compared to in the absence of treatment with sulfide. Similarly, the amount of angiogenesis may be increased at least two-fold, at least three-fold, at least four-fold, at least five-fold, or at least 10-fold, as compared to in the absence of treatment with sulfide. The amount of angiogenesis may be readily determined using routine assays in the art, including any of those described in the accompanying Examples.

It is understood that in certain conditions, sulfide may be used to initiate angiogenesis, while in other conditions; sulfide may be used to increase or enhance angiogenesis. The term “promote angiogenesis” encompasses both initiating and enhancing or increasing angiogenesis. Thus, for example, in particular embodiments, sulfide (or any other agent described herein) may be used to induce or promote growth, proliferation, or migration of cells associated with angiogenesis, e.g., endothelial cells.

In certain embodiments, methods, compositions, and devices of the present invention are used to treat or prevent any of a variety of diseases and disorders that benefit from stimulation of angiogenesis or an increase in angiogenesis in biological matter. For example, compositions and methods of the present invention may be used to promote, enhance, or increase angiogenesis in biological matter in vitro or ex vivo, e.g., in the culture, storage, or generation of tissue or organs suitable for transplant into an organism such as a mammal. Compositions and methods of the present invention may also be used to promote, enhance, or increase angiogenesis in vivo, e.g., at a wound site or a site within an organism subject to or at risk of ischemia or hypoxia, thereby increasing blood flow and oxygenation to the tissue subject to or at risk of ischemia and reducing or preventing tissue injury at the site.

In particular embodiments, the present invention includes improved compositions and methods for treating or preventing pathological conditions, diseases, and disorders that would benefit from enhanced blood flow. Examples of such conditions include ischemia associated diseases. Examples of ischemia associated diseases include myocardial ischemia, peripheral ischemia, cerebral ischemia, and deep vein thrombosis. Furthermore, in related embodiments, the present invention includes improved compositions and methods of treatment for wound healing, diabetes (e.g., diabetic foot ulcers), ocular disease or eye disorder, cardiac disease, congestive heart failure, myocardial ischemia, peripheral ischemia, lymphatic vascular disorders, coronary artery disease, stroke, angina and peripheral vascular disease. In specific embodiments, the compositions and methods of the invention are used in wound healing or reconstructive surgery.

In one embodiment, the present invention includes a method for treating a condition associated with angiogenesis by administering to a subject in need thereof, or cells, tissue, or an organ obtained from said subject, a composition comprising sulfide in an amount effective for stimulating or increasing angiogenesis. In particular embodiments, the subject is a mammal. In certain embodiments, the sulfide is administered locally, e.g., to a site within the subject that is in need of angiogenesis. Examples of such sites within a subject include wounds and tissue or organs subjected to or at risk of ischemia or hypoxia. In other embodiments, the sulfide is administered systemically. In further embodiments, the sulfide is administered ex vivo to cells, tissue, or an organ obtained from the subject, and the cells, tissue or organ contacted with sulfide is then transplanted back into the subject.

In one embodiment, the present invention provides a method for enhancing the survivability of, and/or reducing damage to, biological material under ischemic or hypoxic conditions, which involve contacting the biological material with an amount of sulfide effective to stimulate or increase angiogenesis.

In one aspect the invention relates to methods for treating a condition associated with angiogenesis by administering to a subject in need thereof a composition comprising sulfide in an amount effective for modulating angiogenesis. As used herein, the term “modulating” encompasses any effect on the amount or quality of angiogenesis. In particular embodiments, modulate includes either increasing or decreasing the amount of angiogenesis. Thus, in certain embodiments, the methods described herein are used to promote, enhance or increase angiogenesis, while in other embodiments, the methods described herein are used to decrease or prevent angiogenesis.

In certain aspects, the invention relates to methods for promoting re-epithelialization or wound healing, treating the pathological effects of diabetes (e.g., diabetic foot ulcers), cardiac disease, congestive heart failure, myocardial ischemia, peripheral ischemia, lymphatic vascular disorders, coronary artery disease, stroke, angina, and peripheral vascular disease.

Induction of angiogenesis is beneficial to patients in several pathological disease states including response to injury, wound healing, myocardial ischemia, coronary artery disease, angina and peripheral vascular diseases. Other disorders associated with angiogenesis function may be age-related macular degeneration, or macular dystrophy.

It has recently been demonstrated that the combination of nitric oxide and sulfide may have either additive or synergistic effects in protecting cells and tissue from injury due to exposure to ischemic or hypoxic conditions (see: e.g., in U.S. Provisional Application Nos. 60/877,051 and 60/897,739). Furthermore, it has been shown that sulfide and nitric oxide counteract undesired side-effects that may result from treatment using either compound alone. Thus, according to certain aspects of the present invention, it is contemplated that combinations of nitric oxide and sulfide are used to promote, induce, or increase angiogenesis, or treat or prevent angiogenesis associated conditions. It is believed that such combinations have increased biological and therapeutic activity as compared to either sulfide or nitric oxide alone. In addition, such combinations have reduced side-effects, allowing the use of higher dosages of either or both sulfide and nitric oxide, as compared to when these agents are used alone.

Accordingly, in certain embodiments, the methods described above may be performed using a combination of sulfide and nitric oxide. Thus, in particular embodiments, the present invention provides a method of promoting, increasing, or enhancing angiogenesis comprising contacting biological material with a combination of sulfide and nitric oxide. Similarly, in specific embodiments, the present invention includes a method of treating or preventing an angiogenesis associated condition comprising contacting a subject, or biological matter obtained from a subject, with a combination of sulfide and nitric oxide.

A variety of agents have previously been identified that promote, enhance, or increase angiogenesis, i.e., angiogenesis-inducing agents. Examples of such agents include, but are not limited to, acidic and basic FGF, vascular endothelial growth factor (VEGF), TGFs (TGFα and TGFβ), TNF-α, HGF, angiogenesis factor A, endothelial cell stimulating angiogenesis factor (ESAF) and placental derived growth factor (PDGF).

The present invention further contemplates using sulfide in combination with one or more other angiogenesis-induclng agents. In certain embodiments, nitric oxide is also used in combination with sulfide and one or more other angiogenesis-inducing agents.

Thus, in particular embodiments, the present invention provides a method of promoting, increasing, or enhancing angiogenesis comprising contacting biological material with a combination of sulfide and one or more other angiogenesis-inducing agents. Similarly, in specific embodiments, the present invention includes a method of treating or preventing an angiogenesis associated condition comprising contacting a subject, or biological matter obtained from a subject, with a combination of sulfide and one or more other angiogenesis-induclng agents. Any of these methods may further include contacting the subject (or biological material) with nitric oxide.

When sulfide is used in combination with nitric oxide and/or one or more other angiogenesis-inducing agents, the agents (sulfide, nitric oxide, and other angiogenesis-inducing agents) may be administered simultaneously or in any order. The time periods during which a biological material is exposed to or contacted with sulfide, nitric oxide, and/or one or more other angiogenesis-inducing agent may overlap or be discrete.

### Nitric Oxide and Sulfide Compositions and Formulations

The methods of the present invention may be practiced using a variety of different formulations of nitric oxide and sulfide, including both gas and liquid formulations of each, as well as gas and liquid coformulations comprising both nitric oxide and sulfide. In particular embodiments, any of the following formulations of nitric oxide or sulfide are used.

### Nitric Oxide Formulations and Methods of Manufacture

Nitric oxide may be administered as either a gas or a liquid. In addition, nitric oxide may be directly administered or provided in the form of a prodrug, metabolite or analog, including prodrug forms that release nitric oxide (see U.S. Pat. No. 7,122,529). For instance, a nitric oxide producing compound, composition or substance may undergo a thermal, chemical, ultrasonic, electrochemical, metabolic or other reaction, or a combination of such reactions, to produce or provide nitric oxide, or to produce its chemical or biological effects. Thus, certain embodiments of the present invention include various nitric oxide and nitric oxide prodrugs, including any nitric oxide producing compound, composition or substance. Certain embodiments of the present invention are directed to nitric oxide precursors and catalysts, such as L-arginine, and analogs and derivatives thereof, and nitric oxide synthases (NOS), and mutants/variants thereof.

Various embodiments of the present invention are directed to nitric oxide donors or analogs, which generally donate nitric oxide or a related redox species and more generally provide nitric oxide bioactivity. Examples of nitric oxide donors or analogs include ethyl nitrite, diethylamine NONOate, diethylamine NONOate/AM, spermine NONOate, nitroglycerin, nitroprusside, NOC compounds, NOR compounds, organic nitrates (e.g., glycerin trinitrate), nitrites, furoxan derivatives, N-hydroxy (M-nitrosamine) and perfluorocarbons that have been saturated with NO or a hydrophobic NO donor.

Additional examples of nitric oxide donors or analogs include S-nitroso, O-nitroso, C-nitroso and N-nitroso compounds and nitro derivatives thereof, such as S-nitrosoglutathione, S-nitrosothiols, nitroso-N-acetylpenicillamine, S-nitroso-cysteine and ethyl ester thereof, S-nitroso cysteinyl glycine, S-nitroso-gamma-methyl-L-homocysteine, S-nitroso-L-homocysteine, S-nitroso-gamma-thio-L-leucine, S-nitroso-delta-thio-L-leueine, S-nitrosoalbumin, S-Nitroso-N-penicillamine (SNAP), glyco-SNAPs, fructose-SNAP-1. Further examples of nitric oxide donors or analogs include metal NO complexes, isosorbide mononitrate, isosorbide dinitrate, molsodomines such as Sin-1, streptozotocin, dephostatin, 1,3-(nitrooxymethyl)phenyl 2-hydroxybenzoate and related compounds (see U.S. Pat. No. 6,538,033); NO complexes with cardiovascular amines, such as angiopeptin, heparin, and hirudin, arginine, and peptides with an RGD sequence (See U.S. Pat. No. 5,482,925); diazeniumdiolates such as ionic diazeniumdiolates, O-derivatised diazeniumdiolates, C-based diazeniumdiolates, and polymer based diazeniumdiolates.

In certain embodiments, formulations of nitric oxide suitable for administration according to embodiments of the present invention are liquid solutions. Such solutions may comprise water, dextrose, or saline, polymer-bound compositions dissolved in diluents; other aqueous or nonaqueous solvents, such as vegetable oil, synthetic aliphatic acid glycerides, esters of higher aliphatic acids or propylene glycol, including the addition of conventional additives such as solubilizers, isotonic agents, suspending agents, emulsifying agents, stabilizers and preservatives; capsules, sachets or tablets, each containing a predetermined amount of the nitric oxide; solids or granules; suspensions in an appropriate liquid; suitable emulsions; and gases and/or aerosols, for example, as used in inhalation and nebulizer therapy (see, e.g., U.S. Pat Nos. 5,823,180 and 6,314,956).

In particular embodiments, the present invention includes aerosol formulations, which may include aqueous solutions, lipid soluble aqueous solution, and micronized powders. In certain embodiments the aerosol particle size is between about 0.5 micrometers and about 10 micrometers. Aerosols may be generated by a nebulizer or any other appropriate means.

With respect to gas formulations, those compounds/compositions that are either normally gases or have been otherwise converted to gases may be formulated for use by dilution in nitrogen and/or other inert gases and may be administered in admixture with oxygen, hydrogen sulfide, air, and/or any other appropriate gas or combination of multiple gases at a desired ratio. Dilution, for example, to a concentration of 1 to 100 ppm is typically appropriate. In particular embodiments, nitric oxide is used in the range of 10-80 ppm mixed into air.

In one embodiment, nitric oxide and oxygen are generally administered to a patient by diluting a nitrogen-nitric oxide concentrate gas containing about 1000 ppm nitric oxide with oxygen or oxygen-enriched air carrier gas to produce an inhalation gas containing nitric oxide in the desired concentration range (usually about 0.5 to 200 ppm, based on the total volume of the inhalation gas) (see; U.S. Pat. No. 5,692,495).

Polymer-bound compounds/compositions of the present invention may also be used; such compositions are capable of releasing nitric oxide, donors, analogs, precursors, etc., in an aqueous solution and preferably release nitric oxide, etc., under physiological conditions. Any of a wide variety of polymers can be used in the context of the present invention. It is only necessary that the polymer selected is biologically acceptable. Illustrative of polymer suitable for use in the present invention include polyolefins, such as polystyrene, polypropylene, polyethylene, polytetrafluorethylene, polyvinylidene difluoride, and polyvinylchlorlde, polyethylenimine or derivatives thereof, polyethers such as polyethyleneglycol, polyesters such as poly(lactide/glycolide), polyamides such as nylon, polyurethanes, biopolymers such as peptides, proteins, oligonucleotides, antibodies and nucleic acids, starburst dendrimers, and the like.

The amount of the compounds/compositions of the present invention to be used as a therapeutic agent, of course, varies according to the compounds/compositions administered, the type of disorder or condition encountered and the route of administration chosen. A suitable dosage is thought to be about 0.01 to 10.0 mg/kg of body weight/day. The preferred dosage is, of course, that amount just sufficient to treat a particular disorder or condition and would preferably be an amount from about 0.05 to 5.0 mg/kg of body weight/day.

When either nitric oxide or sulfide are administered as gases, a suitable dosage is thought to be between 1 ppm (parts per million) and 1000 ppm, preferentially between 5 ppm and 200 ppm.

### Sulfide Formulations and Methods of Manufacture

Sulfide may be administered as either a gas or a liquid. Accordingly, the present, invention includes the administration of both gaseous and liquid formulations of sulfide or other sulfur-containing compound. A variety of gaseous formulations of sulfide are described, e.g., in U.S. patent application Ser. No. 11/408,734, and liquid compositions of sulfide are described in U.S. patent application Nos. 11/868,348 and 12/023840, and PCT Application Publication No. WO2008/043081. Any of these compounds and liquid compositions of sulfide may be used according to the present invention. In particular embodiments, the present invention is practiced using a liquid pharmaceutical composition of sulfide, including but not limited to any of the compositions described herein.

In particular embodiments, it is specifically contemplated that the sulfide that is provided is hydrogen sulfide (H₂S). However, it is also contemplated that other sulfur containing compounds may be administered instead of hydrogen sulfide. These include, e.g., sodium sulfide, sodium thiomethoxide, cysteamine, sodium thiocyanate, cysteamine-S-phosphate sodium salt, or tetrahydrothiopryan-4-ol.

In certain embodiments, the pharmaceutical composition provides an effective dose of H₂S to provide when administered to a patient a Cₘₐₓ or a steady state plasma concentration of between 1 μM to 10 mM, between about 1 μM to about 1 mM, or between about 10 μM to about 500 μM. In relating dosing of hydrogen sulfide to dosing with sulfide salts, in typical embodiments, the dosing of the salt is based on administering approximately the same sulfur equivalents as the dosing of the H₂S. Appropriate measures will be taken to consider and evaluate levels of sulfur already in the blood.

A gaseous form or salt of H₂S is specifically contemplated in some aspects of the invention. With hydrogen sulfide gas, for example, in some embodiments, the concentration may be from about 0.01 to about 0.5 M (at STP). Typical levels of hydrogen sulfide contemplated for use in accordance with the present invention include values of about 1 to about 150 ppm, about 10 to about 140 ppm, about 20 to about 130 ppm, and about 40 to about 120 ppm, or the equivalent oral, intravenous or transdermal dosage thereof. Other relevant ranges include about 10 to about 80 ppm, about 20 to about 80 ppm, about 10 to about 70 ppm, about 20 to about 70 ppm, about 20 to about 60 ppm, and about 30 to about 60 ppm, or the equivalent oral, intravenous or transdermal thereof. It also is contemplated that, for a given animal in a given time period, the sulfide atmosphere should be reduced to avoid a potentially lethal build up of sulfide in the subject. For example, an initial environmental concentration of 80 ppm may be reduced after 30 min to 60 ppm, followed by further reductions at 1 hr (40 ppm) and 2 hrs (20 ppm).

In other embodiments, a liquid sulfide composition is contemplated. In certain embodiments, the concentration of the chalcogenide, e.g., sulfide, or salt or precursor thereof in a liquid chalcogenide composition of the present invention is about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 mM or M or more or any range derivable therein (at standard temperature and pressure (STP)). In particular embodiments, liquid pharmaceutical compositions of the present invention comprise a sulfide wherein the concentration of sulfide is in the range 1 mM -250 mM.

Liquid pharmaceutical compositions of the present invention may include a sulfur containing compound or salt or precursor thereof in any desired concentration. The concentration may be readily optimized, e.g., depending upon the type of biological matter being treated and the route of administration, so as to deliver an effective amount in a convenient manner and over an appropriate time-frame. In some embodiments, the concentration of sulfur-containing compound or salt or precursor thereof is in the range of 0.001 mM to 5,000 mM, in the range of 1 mM to 1000 mM, in the range of 50 to 500 mM, in the range of 75 to 250 mM, or in the range of 95 mM to 150 mM. In another embodiment, the concentration of sulfide is in the range 10 mM -200 mM. In certain embodiments, the concentration of sulfide is about 80% to about 100% by w/v.

In one embodiment, the pH of a liquid pharmaceutical composition of the present invention is in the range of (5.0-9.0). The pH of the liquid pharmaceutical composition may be adjusted to a physiologically compatible range. For example, in one embodiment, the pH of the liquid pharmaceutical composition is in the range of 6.0-8.5 or 6.5-8.5. In another embodiment, the liquid pharmaceutical compositions of the present invention have a pH in the range of 7.0-8.0.

In one embodiment, methods of preparing liquid pharmaceutical compositions of the present invention further comprise adjusting the osmolarity of the liquid pharmaceutical composition to an osmolarity in the range of 200-400 mOsmol/L. In one embodiment, the osmolarity of the liquid pharmaceutical composition is in the range of 240-360 mOsmol/L or an isotonic range. In one embodiment, the osmolarity of the liquid pharmaceutical composition is in the range of 250-330 mOsmol/L.

In certain embodiments, isotonicity of liquid pharmaceutical compositions is desirable as it results in reduced pain upon administration and minimizes potential hemolytic effects associated with hypertonic or hypotonic compositions.

### Conformations of Nitric Oxide and Sulfide and Methods of Manufacture

The present invention further provides both gas and liquid compositions comprising both nitric oxide and sulfide.

Gas Conformulations

In one embodiment, the present invention provides a gas coformulation comprising gas nitric oxide and gas sulfide. In particular embodiments, the gas coformulation further comprises air.

In one embodiment, the amount of nitric oxide is about the same or exceeds any amount of hydrogen sulfide in the gas mixture. In one embodiment, the atmosphere will be close to 100% NO, but as will be evident to one skilled in the art, the amount of NO may be balanced with hydrogen sulfide gas and/or air. In this context, the ratio of nitric oxide to hydrogen sulfide is preferably 85:15 or greater, 199:1 or greater or 399:1 or greater. In another embodiment, the amount of sulfide is about the same or exceeds any amount of nitric oxide in the gas mixture. In one embodiment, the atmosphere will be close to 100% sulfide, but as will be evident to one skilled in the art, the amount of sulfide may be balanced with nitric oxide gas and/or air. In this context, the ratio of hydrogen sulfide to nitric oxide is preferably 85:15 or greater, 199:1 or greater or 399:1 or greater.

In certain embodiments, the ratio of either sulfide to nitric oxide or nitric oxide to sulfide is about, at least about, or at most about 1:1, 2:1, 2.5:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, 160:1, 170:1, 180:1, 190:1, 200:1, 210:1, 220:1, 230:1, 240:1, 250:1, 260:1, 270:1, 280:1, 290:1, 300:1, 310:1, 320:1, 330:1, 340:1, 350:1, 360:1, 370:1, 380:1, 390:1, 400:1, 410:1, 420:1, 430:1, 440:1, 450:1, 460:1, 470:1, 480:1, 490:1, 500:1 or more, or any range derivable therein.

In some cases, the amount of nitric oxide or sulfide is relative to each other, while in others, one or both are provided as absolute amounts. For example, in some embodiments of the invention, the amount of nitric oxide or sulfide is in terms of “parts per million (ppm),” which is a measure of the parts in volume of nitric oxide or sulfide, respectively, in a million parts of air at standard temperature and pressure of 20° C. and one atmosphere pressure. In one embodiment, the balance of the gas volume is made up with hydrogen sulfide or nitric oxide, respectively. In one embodiment, nitric oxide is included at an effective concentration, and the balance of the gas volume is made up with hydrogen sulfide. Alternatively, the balance of the gas volume may include sulfide at an effective amount and remainder as air. In another embodiment, sulfide is included at an effective concentration, and the balance of the gas volume is made up with nitric oxide. In another embodiment, the balance of the gas volume may include nitric oxide at an effective amount and remainder as air. In specific embodiments, a gas composition includes nitric oxide at a concentration of 1-150 or 10-80 ppm and sulfide at a concentration of 1-150 or 10-80 ppm, with the remainder of the gas volume made up with air. In one embodiment, the amount of nitric oxide to hydrogen sulfide is related in terms of parts per million of nitric oxide balanced with hydrogen sulfide.

In particular embodiments, it is contemplated that the atmosphere to which the biological material is exposed or incubated may be at least 0, 10, 20, 40, 60, 80, 100, or 200, parts per million (ppm) of nitric oxide balanced with hydrogen sulfide and in some cases sulfide mixed with a non-toxic and/or non-reactive gas and/or air

In one embodiment, co-administration of NO and sulfide to biological matter, comprises nitric oxide and sulfide gases formulated separately in pressurized gas cylinders wherein a known concentration of NO or sulfide is mixed with an inert gas (e.g., nitrogen or argon), wherein the ratio of NO to sulfide can be adjusted by mixing of the container contents at various flow rates prior to exposing the biological matter to the mixture of NO and sulfide. The ratio of NO and sulfide may be varied.

In one embodiment, co-administration of NO and sulfide to biological matter, comprises nitric oxide and sulfide gases formulated together in a single pressurized gas cylinder wherein known concentrations of both NO and sulfide are mixed with an inert gas (e.g., nitrogen or argon) and the ratio of NO to sulfide is fixed.

In either embodiment, if is contemplated that the NO/sulfide mixture is further mixed with air or oxygen prior to exposure to the biological matter. Devices that can monitor the absolute concentrations of NO and sulfide and that can blend NO, sulfide, air and oxygen in defined concentrations are known to those skilled in the art and further described herein.

Alternatively, the atmosphere may be expressed in terms of kPa. It is generally understood that 1 million parts=101 kPa at 1 atmosphere. In embodiments of the invention, the environment in which a biological material is incubated or exposed to is about, at least about, or at most about 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.5, 0.90, 0.95, 1.0 kPa or more nitric oxide, or any range derivable therein. As described above, such levels can be balanced with hydrogen sulfide and/or other non-toxic and/or non-reactive gas(es). Also, the atmosphere may be defined in terms of NO levels in kPa units. In certain embodiments, the atmosphere is about, at least about, or at most about 1, 5, 10, 15, 20, 25, 30, 35, 40, 45, 50, 56, 60, 65, 70, 75, 80, 85, 90, 95, 101, 101.3 kPa NO, or any range derivable therein. In particular embodiments, the partial pressure is about or at least about 85, 90, 95, 101, 101.3 kPa NO, or any range derivable therein.

In embodiments of the invention, the environment in which a biological material is incubated or exposed to is about, at least about, or at most about 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.5, 0.90, 0.95, 1.0 kPa or more sulfide, or any range derivable therein. As described above, such levels can be balanced with nitric oxide and/or other non-toxic and/or non-reactive gas(es). Also, the atmosphere may be defined in terms of sulfide levels in kPa units. In certain embodiments, the atmosphere is about, at least about, or at most about 1, 5, 10, 15, 20, 25, 30, 35, 40, 45, 50, 55, 60, 65, 70, 75, 80, 85, 90, 95, 101, 101.3 kPa sulfide, or any range derivable therein. In particular embodiments, the partial pressure is about or at least about 85, 90, 95, 101, 101.3 kPa sulfide, or any range derivable therein.

Liquid Coformulations

The present invention provides liquid formulations or compositions comprising both sulfide and nitric oxide. The present invention also provides methods of preparing such formulations, as demonstrated in the Examples. In certain embodiments, liquid formulations of sulfide are prepared essentially as described in U.S. Provisional Patent Application Nos. 60/849,900 and 80/898,727, and nitric oxide is added to the resulting formulation, e.g., by bubbling nitric oxide gas into the sulfide liquid formulation.

Liquid pharmaceutical compositions of the present invention may include sulfide in any desired concentration. In particular embodiments, the concentration of sulfide is optimized to be therapeutically effective for its intended purpose. In another embodiment, the concentration of sulfide is optimized to be effective in reducing the undesired side-effects of nitric oxide. The concentration may be readily optimized, e.g., depending upon the type of biological matter being treated and the route of administration, so as to deliver an effective amount in a convenient manner and over an appropriate time-frame. In some embodiments, the concentration of sulfide or salt or precursor thereof is in the range of 0.001 mM to 5,000 mM, in the range of 1 my to 1000 mM, in the range of 50 to 500 mM, in the range of 70 to 250 mM, or in the range of 95 mM to 150 mM. The liquid pharmaceutical compositions of the present invention further comprise sulfide wherein the concentration of sulfide is in the range 1 mM -250 mM. In another embodiment, the concentration of sulfide is in the range 10 mM -200 mM.

Liquid pharmaceutical compositions of the present invention may include nitric oxide in any desired concentration. In particular embodiments, the concentration of nitric oxide is optimized to be therapeutically effective for its intended purpose. In another embodiment, the concentration of nitric oxide is optimized to be effective in reducing the undesired side-effects of sulfide. The concentration may be readily optimized, e.g., depending upon the type of biological matter being treated and the route of administration, so as to deliver an effective amount in a convenient manner and over an appropriate time-frame. In one embodiment, the concentration of nitric oxide is in the range of 1 μM -3 mM in the pharmaceutical composition. In one embodiment, the concentration of nitric oxide is in the range of 10 μM -2 mM in the pharmaceutical composition. In one particular embodiment, the concentration of nitric oxide is in the range of 100 μM -2 mM in the pharmaceutical composition.

In various embodiments, the liquid composition is prepared in a liquid or solution in which the oxygen has been reduced prior to contacting the liquid or solution with nitric oxide or sulfide. Examples of suitable liquids include water and phosphate-buffered saline. Particular embodiments of the present invention further comprise limiting oxygen content in each aspect of manufacturing and storage of the pharmaceutical composition. In one embodiment, oxygen is measured in the range of 0 μM -5 μM in the pharmaceutical composition. In one embodiment, oxygen is measured in the range of 0 μM -3 μM in the pharmaceutical composition. In one embodiment, oxygen is measured in the range of 0.001 μM -0.1 μM in the pharmaceutical composition. In one embodiment, oxygen is measured in the range of 0.1 82 M -1 μM in the pharmaceutical composition.

Nitric oxide and sulfide are not stable in the presence of oxygen due to their ability to react chemically with oxygen, leading to their oxidation and chemical transformation. Accordingly, oxygen may be removed from liquids or solutions using methods known in the art, including, but not limited to, application of negative pressure (vacuum degasing) to the liquid or solution, or contacting the solution or liquid with a reagent which causes oxygen to be bound or “chelated”, effectively removing it from solution. In particular embodiments, oxygen is removed from the coformulations of the present invention.

In one embodiment, a stock solution of sulfide (e.g., 2.5M) is prepared by dissolving Na₂S*9H₂O crystals in deoxygenated water. The stock solution is then diluted into deoxygenated water to produce a Na₂S solution (e.g., 200 mM). Nitric oxide is then bubbled into the Na₂S solution in an oxygen-free environment. The resulting coformuiation may then be pH adjusted to a final pH of 7.0-8.0.

In another embodiment, aqueous nitric oxide is prepared by saturating pure NO gas and hydrolyzing 1 mM 1-hydroxy-2-oxo-3(N-methyl-3-aminoethyl)-3-methyl-1-triazene (NOC-7) in an oxygen-free environment using a modified Saltzman method, essentially as described in Ohkawa et al, Nitric Oxide (2001) 5:516). A solution of aqueous sulfide is prepared by dissolving Na₂S*9H₂O crystals in deoxygenated water (e.g., 200 mM). The aqueous nitric oxide composition is then combined with the aqueous sulfide composition to produce a liquid composition comprising both nitric oxide and sulfide. The pH may be adjusted to a final pH of 7.0-8.0, if desired.

In another embodiment, aqueous nitric oxide is prepared by saturating pure NO gas and hydrolyzing 1 mM 1-hydroxy-2-oxo-3(N-methyl-3-aminoethyl)-3-methyl-1-triazene (NOC-7) in an oxygen-free environment using a modified Saltzman method, essentially as described in Ohkawa et al., Nitric Oxide (2001) 5:515). Hydrogen sulfide gas is then bubbled into the nitric oxide solution. The pH may be adjusted to a final pH of 7.0-8.0, as desired.

In certain embodiments, the liquid formulations are manufactured in a sealed container that contains a vessel to bold the liquid pharmaceutical composition with access ports for pH measurement, addition of gasses, and dispensing without contact to the outside atmosphere. In one embodiment, the vessel is a three neck flask with ground glass fittings. In one embodiment, the vessel is flushed with nitrogen gas or argon gas to minimize oxygen content to a range of 0.00 μM-3 μM.

In certain embodiments, the solution is dispensed from the flask under positive argon pressure into vials or bottles by filling the headspace with argon to the maximum to prevent oxygen to enter the solution. The dispensing vials or bottles are placed in a glove box that is flushed with a constant stream of argon to minimize oxygen to a range of 0.00 μM -0.5 μM and each bottle or vial is flushed with argon before dispensing. The vials and bottles are made of amber glass to enhance stability and are closed with caps lined with Teflon lined silicon or rubber sealed with plastic caps and using a crown-cap crimper to provide an air-tight seal. In one embodiment, the vials and bottles are comprised of borosillcate glass. In one embodiment, the vials and bottles are comprised of silicon dioxide.

In one embodiment, the liquid pharmaceutical composition is stored in an impermeable container. This is particularly desirable when the oxygen has previously been removed from the solution to limit or prevent oxidation of the pharmaceutical or salt or precursor thereof. Additionally, storage in an impermeable container will inhibit the oxidation products of the pharmaceutical gas from the liquid or solution, allowing a constant concentration of the dissolved pharmaceutical to be maintained. Impermeable containers are known to those skilled in the art and include, but are not limited to, “i.v. bags” comprising a gas impermeable construction material, or a sealed glass vial. To prevent exposure to air in the gas-tight storage container, an inert or noble gas, such as nitrogen or argon, may be introduced into the container prior to closure.

In other related embodiments, liquid pharmaceutical compositions are stored in a light-resistant or a light-protective container or vial, such as an amber vial. The composition is preferably packaged in a glass vial, it is preferably filled to a slight over-pressure in an inert atmosphere, e.g., nitrogen, to prevent/slow oxidative breakdown of the composition, and is contained in a form such that ingress of light is prevented, thereby preventing photochemical degradation of the composition. This may be most effectively achieved using an amber vial. Container systems that permit a solution to be stored in an oxygen-free environment are well known as many intravenous solutions are sensitive to oxygen. For example, a glass container that is purged of oxygen during the filling and sealing process may be used. In another embodiment, flexible plastic containers are available that may be enclosed in an overwrap to seal against oxygen. Basically, any container that prevents oxygen from interacting with the liquid pharmaceutical composition may be used, (see: U.S. Pat. No. 6,458,758) In one embodiment, the container includes one or more oxygen scavenger. For example, the oxygen scavenging composition can be applied as a coating or lining upon the inside surface of the product supporting or retaining means to function as a barrier to oxygen permeation (see: U.S. Pat. No. 5,492,742).

### Nitric Oxide and Sulfide Products

The pharmaceutical compositions of the present invention may comprise one or more nitric oxide and/or sulfide products. In various embodiments, one or more nitric oxide or sulfide products is present in an amount less than 20%, less than 10%, less than 6.0%, less than 3.0%, less than 1.0%, less than 0.5%, less than 0.2%, less than 0.1%, less than 0.05%, or less than 0.01 %. As used herein, the term “%” when used without qualification (as with w/v, v/v, or w/w) means % weight-in-volume for solutions of solids in liquids (w/v),

% weight-in-volume for solutions of gases in liquids (w/v), % volume-in-volume for solutions of liquids in liquids (v/v) and weight-in-weight for mixtures of solids and semisolids (w/w) (Remington's Pharmaceutical Sciences (2005); 21^{st} Edition, Troy, David B. Ed, Lippincott, Williams and Wilkins).

In one embodiment, a nitric oxide product is a nitrosothiol. In one embodiment, the nitrosothiol product is present in the range of 0%-20% (w/v). In one embodiment, the nitrosothiol product is in the range of 4.0%-10.0% (w/v). In one embodiment, the nitrosothiol product is in the range of 3.0%-6.0% (w/v). In one embodiment the nitrosothiol product is in the range of 1.0%-3.0% (w/v). In one embodiment, the nitrosothiol product is in the range of 0%-1.0% (w/v).

In one embodiment, the peroxynitrite product is present in the range of 4.0%-10.0% (w/v). In one embodiment, the nitrosothiol product is in the range of 3.0%-6.0% (w/v). In one embodiment the nitrosothiol product is in the range of 1.0%-3.0% (w/v). In one embodiment, the nitrosothiol product is in the range of 0%-1.0% (w/v).

The pharmaceutical composition of the present invention may further comprise sulfide oxidation products. Oxidation products of the present invention include, but are not limited to, sulfite, sulfate, thiosulfate, polysulfides, dithionate, polythionate, and elemental sulfur. In various embodiments, one or more of these oxidation products is present in an amount less than 10%, less than 6.0%, less than 3.0%, less than 1.0%, less than 0.5%, less than 0.2%, less than 0.1%, less than 0.05%, or less than 0.01%.

In one embodiment, the oxidation product, sulfite, is present in the range of 0%-10% (w/v). In one embodiment, the oxidation product, sulfite, is in the range of 3.0%-6.0% (w/v). In one embodiment the oxidation product, sulfite, is in the range of 1.0%-3.0% (w/v). In one embodiment, the oxidation product, sulfite, is in the range of 0%-1.0% (w/V).

In one embodiment, the oxidation product, sulfate, is present in the range of 0%-10.0% (w/v). In one embodiment, the oxidation product, sulfate, is in the range of 3.0%-6.0% (w/v). In one embodiment, the oxidation product, sulfate, is in the range of 1 % to 3.0% (w/v). In one embodiment, the oxidation product, sulfate, is in the range of 0%-1.0% (w/v).

In one embodiment, the oxidation product, thiosulfate, is present in the range of 0%-1G% (w/v). In another embodiment, the oxidation product, thiosulfate, is in the range of 3.0%-6.0% (w/v). In another embodiment, the oxidation product, thiosulfate, is in the range of 1.0%-3.0% (w/v). In another embodiment, the oxidation product, thiosulfate, is in the range of 0%-1.0% (w/v).

In one embodiment, the oxidation products include polysulfides present in the range of (0%-10% (w/v). In one embodiment, the oxidation products, polysulfides, are in the range of 3.0%-8.0% (w/v). In one embodiment the oxidation products, polysulfides, are in the range of 1.0%-3.0% (w/v). In one embodiment, the oxidation products, polysulfides, are in the range of 0%-1.0% (w/v).

In one embodiment, the oxidation product, dithionate, is present in the range of 0%-10% (w/V). In one embodiment, the oxidation product, dithionate, is in the range of 3.0%-6.0% (w/v). In one embodiment the oxidation product, dithionate, is in the range of 1.0%-3.0% (w/v). In one embodiment, the oxidation product, dithionate, in the range of 0%-1.0% (w/v).

In one embodiment, the oxidation product, polythionate, is present in the range of 0%-10% (w/v). In one embodiment, the oxidation product, polythionate, is in the range of 3.0%-6.0% (w/v). In one embodiment the oxidation product, polythionate, is in the range of 1.0% -3.0% (w/v). In one embodiment, the oxidation product, polythionate, is in the range of 0% -1.0% (w/v).

In one embodiment, the oxidation product, elemental sulfur, is present in the range of 0%-10% (w/v). In one embodiment, the oxidation product, elemental sulfur, is in the range of 3.0% -6.0% (w/v). In one embodiment the oxidation product, elemental sulfur, is in the range of 1.0%-3.0% (w/v). In one embodiment, the oxidation product, elemental sulfur, is present in the range of 0% -1.0% (w/v).

### Pharmaceutical Compositions and Routes of Delivery

The present invention contemplates the administration of gas and liquid compositions described herein to patients, including humans and other mammals. Therefore, the present invention includes all pharmaceutical compositions comprising either or both nitric oxide and sulfide. In particular embodiments, a liquid pharmaceutical composition of sulfide is prepared as described in the accompanying Examples. In one particular embodiment, a stable liquid pharmaceutical composition of sulfide is prepared by dissolving one equivalent of hydrogen sulfide gas info one equivalent of sodium hydroxide solution, wherein said composition has a pH in the range of 6.5 to 8.5, wherein said composition has an osmolarity in the range of 250-330 mOsmol/L, wherein said composition has an oxygen content of less than or equal to 5 μM, and wherein said composition comprises oxidation products are the range of 0%-3.0% (w/v) after storage for three months.

In some embodiments, compositions of the present invention are pharmaceutically acceptable parenteral formulations (e.g., intravenous, intraarterial, subcutaneous, intramuscular, intracistemal, intraperitoneal, and intradermal) dosage forms. In other embodiments, liquid pharmaceutical compositions are formulated for oral, nasal (inhalation or aerosol), nebulizer, buccal, or topical administration dosage forms.

In various embodiments, methods of the present invention include deliver by any suitable route. Accordingly, in certain embodiments, methods of the invention include and compositions of the present invention may be administered through inhalation, injection, catheterization, immersion, lavage, perfusion, topical application, absorption, adsorption, oral administration, intravenously, intradermally, intraarterially, intraperitoneally, intralesionally, intracranially, intraarticularty, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbslically, intraocularally, orally, topically, locally, by inhalation, by injection, by infusion, by continuous infusion, by localized perfusion, via a catheter, or via a lavage.

In certain embodiments, it may be desirable to deliver the sulfide formulation topically, e.g., for localized delivery, e.g., to facilitate wound healing. Topical application can be accomplished by use of a biocompatible gel, which may be provided in the form of a patch, or by use of a cream, foam, and the like. Several gels, patches, creams, foams, and the like appropriate for application to wounds can be modified for delivery of angiogenic compositions according to the invention (see, e.g., U.S. Pat. Nos. 5,853,749; 5,844,013; 5,804,213; 5,770,229; and the like). In general, topical administration is accomplished using a carrier such as a hydrophllic colloid or other material that provides a moist environment.

In some embodiments, the topical formulation is a combination of sulfide and nitric oxide.

The parenteral liquid compositions may be buffered to a certain pH to enhance the solubility of the nitric oxide and/or sulfide or to influence the ionization state of the nitric oxide and/or sulfide. In addition, the compositions described herein may further include the addition of one or more of a metal chelator, a free radical scavenger, and/or a reducing agent.

The compositions and formulations of the present invention are, in certain embodiments, formulated for pharmaceutical use. Accordingly, they may include a variety of different pharmaceutical excipients and carriers, and may be formulated for pharmaceutical use as described, e.g., in U.S. Provisional Application No. 60/868,727 and U.S. Provisional Patent Application No. 60/896,739.

The effective concentration of nitric oxide gas to achieve a therapeutic effect in a human depends on the dosage form and route of administration. For inhalation, in some embodiments effective concentrations are in the range of 5 ppm to 100 ppm, delivered intermittently or continuously. The effective concentration of liquid nitric oxide formulations is in the range of 0.01 mg/kg to 100 mg/kg, preferably 0.1 mg/kg to 10 mg/kg, delivered continuously or intermittently.

The effective concentration of hydrogen sulfide to achieve a therapeutic effect in a human depends on the dosage form and route of administration. For inhalation, in some embodiments, effective concentrations are in the range of 10 ppm to 500 ppm, delivered intermittently or continuously. The effective concentration of liquid sulfide formulations are in the range of 0.01 mg/kg to 100 mg/kg, preferably 0.1 mg/kg to 10 mg/kg, delivered continuously or intermittently.

The effective concentration of hydrogen sulfide to achieve stasis in a human depends on the dosage form and route of administration. For inhalation, in some embodiments, effective concentrations are in the range of 50 ppm to 500 ppm, delivered intermittently or continuously.

### Devices and Kits for the Preparation and Administration of Nitric Oxide and Sulfide

In certain embodiments, methods of the invention are practiced using a specific delivery device or apparatus. Any method discussed herein can be implemented with any device for delivery or administration including, but not limited to, those discussed herein or described in PCT application WO/2008/113914. In one embodiment, hydrogen sulfide gas or nitric oxide gas or hydrogen sulfide gas and nitric oxide gas may be administered and levels monitored by gas delivery systems well known in the art (see, e.g., U.S. Pat. No. 6,109,260; 6,581,592; 6,089,229; 6,125,846; 5,839,433; 5,692,495; 8,164,276; 5,732,693; 5,558,083). It is contemplated that either hydrogen sulfide gas or nitric oxide gas or hydrogen sulfide gas and nitric oxide gas may be administered by the gas delivery devices described herein.

In certain embodiments, gas delivery devices described in US 2005/013625, US 2005/0147692, or US 2005/0170019 may be used to administer gas to a cell, tissue organ, organ system or organism. In one embodiment, gases may be administered using an implantable medical device for controlled release of gaseous agents (see; U.S. Pat. No. 7,122,027).

Additional exemplary devices include electrohydrodynamic (END) aerosol delivery devices and EHD aerosol devices use electrical energy to aerosolize liquid drag solutions or suspensions (see e.g., Noakes of al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807. EHD aerosol devices may more efficiently deliver drags to the lung than existing pulmonary delivery technologies.

In certain embodiments, methods of the present invention are practiced using a nebulizer. Nebulizers create aerosols from liquid drag formulations by using, for example, ultrasonic energy to form fine particles that may be readily inhaled. Examples of nebulizers include devices supplied by Sheffield/Systemic Pulmonary Delivery Ltd. (See, Armer et al, U.S. Pat. No. 5,954,047; van der Linden et al, U.S. Pat. No. 5,950,019; van der Linden et al., U.S. Pat. No. 5,970,974), Intal nebulizer solution by Aventis, (e.g., world wide web at fda.gov/medwatch/SAFETY/2004/feb_PI/Intal_Nebulizer_PI.pdf).

For administration of a gas directly to the lungs by inhalation, various delivery methods currently available in the market for delivering oxygen may be used. For example, a resuscitator such as an ambu-bag may be employed (see U.S. Pat Nos. 5,988,162 and 4,790,327). An ambu-bag consists of a flexible squeeze bag attached to a face mask, which is used by the physician to introduce air/gas info the casualty's lungs. A portable, handheld medicine delivery device capable producing atomized agents that are adapted to be inhaled through a nebulizer by a patient suffering from a respiratory condition. In addition, such delivery device provides a means wherein the dose of the inhaled agent can be remotely monitored and, if required altered, by a physician or doctor. See U.S. Pat. No. 7,013,894. Delivery of the compound of invention may be accomplished by a method for the delivery of supplemental gas to a person combined with the monitoring of the ventilation of the person with both being accomplished without the use of a sealed face mask such as described in U.S. Pat. No. 6,938,619. All the devices described here may have an exhaust system to bind or neutralize the compound of invention.

In one embodiment, the present invention includes a device for the metered coadministration of nitric oxide and sulfide to a patient, comprising a first compartment containing nitric oxide gas, a second compartment containing sulfide gas, wherein said first and second compartments are attached to a device for mixing the contained nitric oxide and sulfide gas prior to administration to a patient.

In another embodiment, the present invention includes a device for the metered coadministration of nitric oxide and sulfide to a patient, characterized by a gas feed system including a first line feeding nitric oxide, a second line feeding sulfide, a shut-off valve in the first line, a shut-off valve in the second line, wherein the first and second lines are in flow communication with a third line, whereby upon opening both shut-off valves to open flow nitric oxide and sulfide may flow through the first and second lines and into the third line, where they are mixed, and a device for delivering the resulting mixture of nitric oxide and sulfide to the patient, wherein said device is in flow communication with the third line. In particular embodiments, the device further include a fourth line feeding air and a shut-off valve in the fourth line, wherein the fourth line is in flow communication with the third line, whereby upon opening all shut-off valves to open flow nitric oxide, sulfide, and air may flow through the first, second, and third lines and into the third line, where they are mixed.

### EXAMPLE 1

### A Liquid Formulation of Sulfide Stimulates Angiogenesis in the Chick Chorioallantoic Membrane (CAM) Assay

The ability of a liquid formulation of sulfide to promote neovascularization in an in vivo model was examined using the CAM assay. Five to 10 day-old, Leghorn chicken fertilized eggs were incubated for four days at 37° C. Using a candling lamp in the dark, a small hole was punctured in the shell with a hypodermic needle in the area that concealed the air sac. A second hole was punctured in the shell on the broadside of the egg directly over the non-vascularized area of the embryonic membrane. A false or pseudo air sac was created beneath the second hole by the application of negative pressure to the first hole, causing the chorioallantoic membrane (CAM) to separate from the shell. An opening or window, approximately 1.0 cm² (restricted by a plastic ring), was cut into the shell over the dropped CAM which allowed direct access to the underlying CAM.

At day four, following exposure of the CAM, either vehicle or test article (liquid formulation of sulfide) was administered in concentrations of 0.24, 2.4, 24, or 240 pmol/cm² and incubated at 37° C. for 48 hours. The liquid formulation of sulfide test article was prepared by dissolving hydrogen sulfide in NaOH solution under oxygen-free conditions and sterile filtration, essentially as described in Example 5 (Liquid Pharmaceutical Composition IV). The formulation contained 60 mM NaCl, 90 mM NaOH, 98 mM sulfide, and 4.88 μM polysulfide. The formulation had a pH of 7.81, a mOsm/l of 290, and OD370 of 0.1.

Forty-eight hours after treatment, the CAMs were fixed in situ, excised from the eggs, placed on slides, and left to air-dry. A stereoscope equipped with a digital camera was used to photograph the treated CAMs and a total length of the vessels was measured using image analysis software. Assays for each test sample were completed in triplicate. Ten eggs per data point were tested.

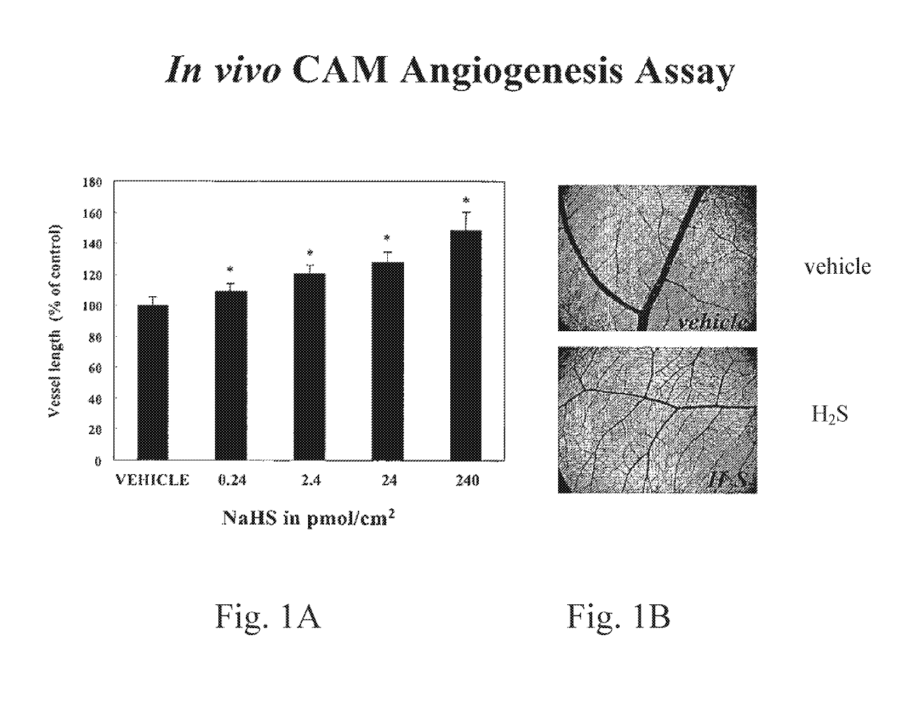

As shown in FIG. 1A, the total length of vessels was increased in a dose-responsive manner upon treatment with the liquid sulfide formulation, as compared to treatment with vehicle alone. In addition, the CAM vascular network appeared more developed after treatment with the liquid sulfide formulation as compared to vehicle (FIG. 1B). These data demonstrate that liquid sulfide promotes blood vessel formation in vivo.

### EXAMPLE 2

### A Liquid Formulation of Sulfide Stimulates Angiogenesis in the Human Umbilical Vein Endothelial Cell (HUVEC) Tube Formation Assay

The ability of a liquid formulation of sulfide to promote angiogenesis was further examined by observing HUVEC tube formation. Matrigel®, a solubilized basement membrane preparation extracted from EHS mouse sarcoma, a tumor rich in extracellular matrix (ECM) proteins (laminin, collagen IV, heparin sulfate proteoglycans, and entactin) was used to coat the wells of 98-well tissue culture plates (0.04 ml/well) and left to solidify for one hour at 37° C. Approximately 15,000 HUVECs were then suspended in 0.15 ml of M199 media supplemented with 5% fetal calf serum and added to each well. Either vehicle or the liquid hydrogen sulfide test article described in Example 1 (60 μM) were added to the corresponding wells simultaneously with the cells. After six hours of incubation at 37° C., the medium was removed, the cells were fixed, and the length of structures that resemble capillary cords was measured in the total area of the wells using image analysis softer as previously described (Loutrari et al., JPET 2004, 311:588-575). The tube-like network as percent of control was measured.

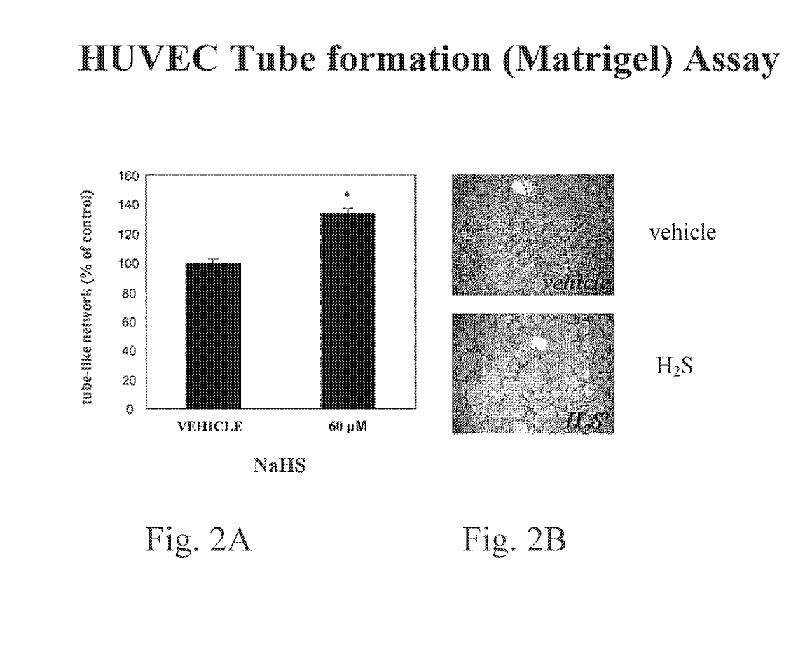

As shown in FIG. 2A, the length of the tube network was significantly greater in HUVECs treated with hydrogen sulfide as compared to those treated with control vehicle. In addition, photomicrographs of the different HUVEC cultures showed an increased amount of tube-like structures upon treatment with the liquid formulation of sulfide as compared to control vehicle (FIG. 2B). These results demonstrate that hydrogen sulfide promotes the formation of blood vessels from endothelial cells.

### EXAMPLE 3

### A Liquid Formulation of Sulfide Stimulates Proliferation of Human Umbilical Vein Endothelial Cells (HUVEC)

The ability of a liquid formulation of sulfide to stimulate proliferation of HUVECs was also examined. Isolated and cultured HUVECs were seeded on rat tail type 1 collagen coated wells at 2000 cells/well in a 96 well plate. The mean number of cells per dish for each condition was then calculated either by MTT assay or by direct cell counting. Twenty-four hours after seeding, the cells were treated with fresh media containing different concentrations of the liquid sulfide test compound described in Example 1 (6 μM, 60 μM, or 600 μM) or vehicle and further cultured for 24 hours. The proliferation rates in 3-D collagen cultures were assessed as a percentage of baseline measurement. The experiments were performed in duplicate at passage two, using 4-6 well each time.

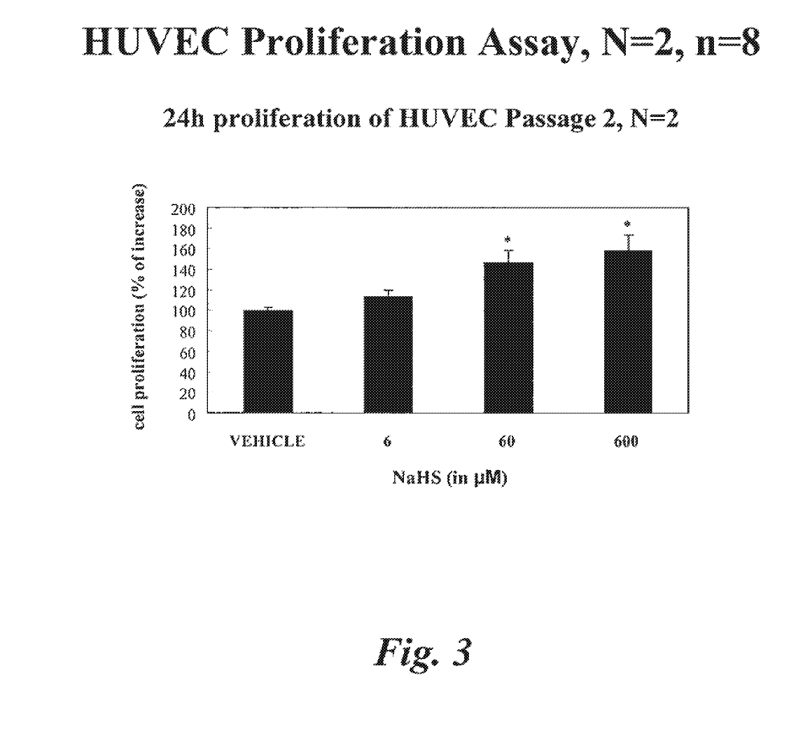

As shown in FIG. 3, the proliferation rates of HUVECs in 3-D collagen culture was increased in a dose-dependent manner upon treatment with the liquid sulfide formulation as compared to treatment with vehicle alone. These data demonstrate that the liquid sulfide formulation significantly enhanced the proliferation of HUVECs, further establishing its ability to promote neovascularization.

### EXAMPLE 4

### Hydrogen Sulfide and Nitric Oxide Stimulate Angiogenesis

To determine the effect on angiogenesis of combination treatment with nitric oxide in addition to hydrogen sulfide, the angiogenesis assays described in examples 1-3 are performed wherein cells are treated with control vehicle, hydrogen sulfide alone, nitric oxide alone, or a combination of hydrogen sulfide and nitric oxide. The combination of hydrogen sulfide and nitric oxide should result in an increase in CAM neovascularization, HUVEC tube formation, and HUVEC proliferation greater than the increase resulting from treatment with either hydrogen sulfide or nitric oxide alone.

### EXAMPLE 5

### Methods of Manufacturing Liquid Sulfide Compositions

Liquid pharmaceutical sulfide compositions were prepared as described below.

Stock solutions were prepared using deoxygenated water. The water was deoxygenated by removing air under vacuum and dissolving with compressed nitrogen (99.99%) for 30 minutes. A saturated stock solution of 2.5 M Na₂S was prepared from Na₂S*9H2O crystals (Fisher #5425) that were rinsed with oxygen-free, distilled, deionized water. This stock was stored tightly sealed and protected from light. A 220 mM stock solution of HCl was prepared by dilution of concentrated acid (Fisher # A144-212) and deoxygenated by dissolving with compressed nitrogen.

Liquid pharmaceutical compositions were prepared in a fume hood in a basic glove box filled with nitrogen gas to yield an oxygen-free environment. The reactor with pH meter, bubbler and stirrer were in the glove box. Oxygen levels in the glove box were monitored with an oxygen meter (Mettler-Toledo) with a sensitivity level of 0.03 μM. Methods of preparing the liquid pharmaceutical compositions of the present invention include limiting oxygen content in each aspect of manufacturing and storage of the pharmaceutical composition where oxygen is measured in the range of 0 μM-5 μM in the pharmaceutical composition.

Liquid pharmaceutical compositions were prepared in a three-neck flask (Wilmad Labs) with each opening fitted with ground glass fittings having the following features:

a. A universal adapter with a plastic cap with a central orifice and o-ring. This adapter was fitted with a pH probe and sealed by the O-ring.

b. Universal adapter with a hose connector and a plastic cap with a central orifice and O-ring. This adapter was fitted with a gas dispersion tube with a glass frit. The dispersion tube was connected to a compressed gas cylinder and used to deoxygenate the solution by dissolving with compressed nitrogen and to neutralize the pH with a mixture of H₂S and nitrogen. The hose connector was fitted with a plastic tube to allow pressure to escape. These two connections were reversed to dispense the contents of the flask under positive nitrogen pressure.

c. The third neck was sealed with a ground glass stopper and used to add Na₂S solution or water to the flask.

### Liquid Pharmaceutical Composition I—Na₂S Nonahydrate

Liquid Pharmaceutical Composition I was prepared with the following steps:

a. Oxygen-free distilled, deionized water was added to a three neck flask and deoxygenated by dissolving with nitrogen for 30 minutes while stirring.

b. 2.5 M Na₂S Stock was added to yield a 200 mM Na₂S solution.

c. The 200 mM Na₂S Solution was bubbled with compressed nitrogen for 15 minutes while stirring.

d. 220 mM HCl was added until a final pH of 7.8-8.0 while dissolving with compressed nitrogen and stirring.

e. Deoxygenated deioinlzed water was added to give a final concentration of 100 mM Na₂S.

### Liquid Pharmaceutical Composition II—Na₂S Nonahydrate

Liquid Pharmaceutical Composition II was prepared with the following steps:

a. Deionized, oxygen-free water was added to the three neck flask and deoxygenated by dissolving with nitrogen for 30 minutes while stirring.

b. 2.5 M Na₂S Stock was added to yield a 100 mM Na₂S solution.

c. The 100 mM Na₂S Solution was bubbled with compressed nitrogen for 15 minutes while stirring.

d. The solution was bubbled with a 50/50 mixture of compressed nitrogen and CO₂ (99.9%) until a pH of 7.8 was reached.

Liquid Pharmaceutical Composition III—Na₂S with H₂S and Nitrogen

Liquid Pharmaceutical Composition III was prepared with the following steps:

a. Deionized, oxygen-free water was added to the three neck flask and deoxygenated by dissolving with nitrogen for 30 minutes while stirring.

b. 2.5 M Na₂S Stock was added to yield a 100 mM Na₂S solution.

c. The 100 mM Na₂S Solution was bubbled with compressed nitrogen for 15 minutes while stirring.

d. The solution was bubbled with a 50/50 mixture of compressed nitrogen and H₂S until a pH of 8.2 was reached. This resulted in a final concentration of 90 mM sulfide.

### Liquid Pharmaceutical Composition IV—H₂S

The final sulfide concentration of Liquid Pharmaceutical Composition IV was determined by the initial concentration of NaOH. Liquid Pharmaceutical Composition IV was prepared with the following steps:

a. NaOH in a range of 5 mM to 500 mM solution was added to the three neck flask with additives (DTPA, antioxidants) (FIG. 1.)

b. The solution was deoxygenated by bubbling with argon at 5 psi for 15 minutes while stirring.

c. H₂S was bubbled through the solution while stirring until pH was reduced to 7.7 (or a range of 7.8 to 7.8).

d. The headspace in the flask was flushed with argon.

e. Amber dispensing bottles or vials were placed in a glove box that was flushed with a constant stream of argon and each bottle or vial was flushed with argon.

f. The formulation was dispensed under argon to maintain an oxygen-free environment.

The stability of the solution was monitored by measurement of sulfide concentration, pH, and absorbance spectrum (polysulfide formation). Additional assays were performed to monitor oxidation products which include sulfite, sulfate, thiosulfate, and elemental sulfur.

Liquid pharmaceutical compositions were dispensed within the sealed Glove box, from the three-necked flask under positive nitrogen pressure. Amber vials or amber bottles were filled to a slight over-pressure in an inert atmosphere argon or nitrogen to prevent/slow oxidative breakdown of the liquid pharmaceutical compositions, and sealed with plastic caps with Teflon/silicon liners or plastic caps with central Teflon lined silicon septa using a crown-cap crimper (Aldrich Z112976) to provide an air-tight seal.

A liquid pharmaceutical composition of sodium sulfide (Liquid Pharmaceutical Composition IV) was prepared that met Good Manufacturing Practices (GMP) acceptance criteria, including concentration, pH, and osmolality, after storage at various commercially acceptable temperatures and durations of time.

### EXAMPLE 6

### Methods of Manufacturing NO in a Pharmaceutically Acceptable Buffer

Two methods for preparing an aqueous formulation of NO are described (see, Ohkawa et al., Nitric Oxide, (2001) 5:515).

According to one method, a 100-ml NO solution in 0.1M phosphate buffer (pH 7.4) was prepared using pure NO gas. NO₂ contamination was minimized. NO gas was purified by a column with a KOH pellet to remove NO₂ in the NO gas tank generated by the dismutation reaction: 3NO→NO₂+N₂O before introduction into the buffer. A column of sodium hydrosulfite on glass wool was attached to avoid exposure of the flask content to atmospheric oxygen. Nitrogen gas was purged to remove NO in the headspace of the flask to avoid conversion of gaseous NO into NO₂ in contact with atmospheric oxygen.

The following five steps were then followed: (1) 0.1 M phosphate buffer (pH 7.4) (100 ml) was placed in the flask and the flask was tightly sealed with a silicone stopper; (2) the solution was kept at 20° C. and gently stirred; (3) nitrogen gas was introduced through the cock at 70 ml/min for 3 h; (4) NO gas was introduced through the cock at 10 ml/min for 17 min; and (5) for determination of the nitrogen oxide species in the aqueous solution, 1.0 ml of the solution was withdrawn by means of a gas-tight syringe through a silicone stopper. For determination of the nitrogen oxide species in the aqueous solution generated in contact with oxygen, the silicone stopper was removed from the flask and 1.0 ml of the solution was withdrawn after keeping the solution at 20° C. for the indicated period under the aerobic conditions.

A second method of manufacture used NOC-7, which releases 2 equivalent amounts of NO in a neutral solution. A 100-ml NO solution in 0.1 M phosphate buffer (pH 7.4) was prepared from NOC-7. The first three steps were followed the same as described in the foregoing, except that the volume of the phosphate buffer was 90 ml, and the temperature of the flask was maintained at 37° C. During a fourth step, a 10-ml solution of 10 mM NOC-7 in 0.1 M NaOH, which had been deoxygenated by purging nitrogen gas, was introduced by means of a gas-tight syringe through the silicon stopper, and the mixture was maintained at 37° C. for 1 h, after which the temperature of the mixture was made at 20° C. Step 5 was the same as described in the foregoing.

### EXAMPLE 7

### Preparation of Pharmaceutical Compositions Comprising Nitric Oxide and Hydrogen Sulfide

Liquid pharmaceutical compositions of comprising both nitric oxide and hydrogen sulfide are prepared according to the methods described herein.

### Method of Manufacture

In one embodiment, liquid pharmaceutical compositions are prepared in a fume hood in a basic glove box filled with nitrogen gas to yield an oxygen-free environment. The reactor with pH meter, bubbler and stirrer are in the glove box. Oxygen levels in the glove box should be monitored with an oxygen meter (Mettler-Toledo) with a sensitivity level of 0.03 μM. Methods of preparing the liquid pharmaceutical compositions of the present invention include limiting oxygen content in each aspect of manufacturing and storage of the pharmaceutical composition where oxygen is measured in the range of 0 μM-5 μM in the pharmaceutical composition.

Liquid pharmaceutical compositions are prepared in a three-neck flask (Wilmad Labs) with each opening fitted with ground glass fittings having the following features:

a. A universal adapter with a plastic cap with a central orifice and o-ring. This adapter is fitted with a pH probe and sealed by the O-ring.

b. Universal adapter with a hose connector and a plastic cap with a central orifice and O-ring. This adapter is fitted with a gas dispersion tube with a glass frit. The dispersion tube will be connected to a compressed gas cylinder and used to deoxygenate the solution by dissolving with compressed nitrogen and to neutralize the pH with a mixture of nitric oxide, H₂S and nitrogen. The hose connector will be fitted with a plastic tube to allow pressure to escape. These two connections are reversed to dispense the contents of the flask under positive nitrogen pressure.

c. The third neck is sealed with a ground glass stopper and used to add Na₂S solution or water to the flask.

### Dispensing and Storage

Liquid pharmaceutical compositions are dispensed within the sealed Glove box, from the three-necked flask under positive nitrogen pressure. Amber vials or amber bottles are filled to a slight over-pressure in an inert atmosphere argon or nitrogen to prevent/slow oxidative breakdown of the liquid pharmaceutical compositions, and sealed with plastic caps with Teflon/silicon liners or plastic caps with central Teflon lined silicon septa using a crown-cap crimper (Aldrich Z112976) to provide an air-tight seal.

### Composition 1: Hydrogen Sulfide Liquid and Nitric Oxide Gas

In this prophetic example, the novel composition comprises a combination of nitric oxide gas and hydrogen sulfide liquid and is prepared as follows. pH of 7.0 to 8.0 is suitable to maintain a sulfide concentration in the composition.

### Starting Materials

Nitric oxide gas: Various methods for the manufacture of nitric oxide for pharmaceutical administration exist. One process for the manufacture of nitric oxide results in the production of a gaseous nitric oxide product which contains little or no nitrous oxide (see: U.S. Pat. No. 5,670,127).

H₂S Liquid composition: Stock solutions are prepared using deoxygenated water. The water is deoxygenated by removing air under vacuum and dissolving with compressed nitrogen (99.99%) for 30 minutes. A saturated stock solution of 2.5 M Na₂S is prepared from: Na₂*9H2O crystals (Fisher #5425) that are rinsed with oxygen-free, distilled, deionized water. This stock is stored tightly sealed and protected from light. A 220 mM stock solution of HCl can be prepared by dilution of concentrated acid (Fisher # A144-212) and deoxygenated by dissolving with compressed nitrogen.

### Steps

1. Oxygen-free distilled, deionized water is added to a three neck flask and deoxygenated by dissolving with nitrogen for 30 minutes while stirring.

2. 2.5 M Na₂S Stock is added to yield a 200 mM Na₂S solution.

3. The 200 mM Na₂S Solution is bubbled with compressed nitrogen for 15 minutes while stirring.

4. Nitric oxide gas is bubbled into the Na₂S solution in an oxygen free environment.

5. pH is adjusted to a final pH of 7.0-8.0, while dissolving with compressed nitrogen and stirring.

### Composition 2: Nitric Oxide Liquid and Hydrogen Sulfide Liquid

### Starting Materials

Nitric oxide liquid composition: In one embodiment, aqueous nitric oxide is prepared by saturating pure NO gas and hydrolyzing 1 mM 1-hydroxy-2-oxo-3(N-methyl-3-aminoethyl)-3-methyl-1 -triazene (NOC-7), in an oxygen-free environment using a modified Saltzman method (see: Ohkawa et al., Nitric Oxide, (2001) 5:515).

H₂S Liquid composition: Stock solutions are prepared using deoxygenated water. The water is deoxygenated by removing air under vacuum and dissolving with compressed nitrogen (99.99%) for 30 minutes. A saturated stock solution of 2.5 M Na₂S will be prepared from Na₂S*9H2O crystals (Fisher #5425) that are rinsed with oxygen-free, distilled, deionized water. This stock is stored tightly sealed and protected from light. A 220 mM stock solution of HCl is prepared by dilution of concentrated acid (Fisher # A144-212) and deoxygenated by dissolving with compressed nitrogen.

### Steps

1. Oxygen-free distilled, deionized water is added to a three neck flask and deoxygenated by dissolving with nitrogen for 30 minutes while stirring.

2. 2.5 Na₂S Stock is added to yield a 200 mM Na₂S solution.

3. The 200 mM Na₂S Solution is bubbled with compressed nitrogen for 15 minutes while stirring.

4. Nitric oxide liquid (prepared as described in the foregoing) is combined with Na₂S solution.

5. pH is adjusted to a final pH of 7.0-8.0, while dissolving with compressed nitrogen and stirring.

Any order may be used to add Na₂S and nitric oxide liquid together.

### A. Composition 3: Nitric Oxide Liquid and Hydrogen Sulfide Gas

Nitric Oxide Liquid Composition: In one embodiment, aqueous nitric oxide is prepared by saturating pure NO gas and hydrolyzing 1 mM 1-hydroxy-2-oxo-3(N-methyl-3-aminoethyl)-3-methyl-1-thiazene (NOC-7), in an oxygen-free environment using a modified Saltzman method (see; Ohkawa et al., Nitric Oxide, (2001) 5:515).

### Steps

1. Oxygen-free distilled, deionized water is added to a three neck flask and deoxygenated by dissolving with nitrogen for 30 minutes while stirring.

2. 2.5 M Na₂S Stock is added to yield a 200 mM Na₂S solution.

3. The 200 mM Na₂S Solution is bubbled with compressed nitrogen for 15 minutes while stirring.

4. Hydrogen sulfide gas is bubbled into the nitric oxide solution in an oxygen-free environment.

5. pH is adjusted to a final pH of 7.0-8.0, while dissolving with compressed nitrogen and stirring.

### EXAMPLE 8

RE-Epithelialization in Rats Was Improved in the Presence of a Liquid Pharmaceutical Sulfide Composition

The Sprague Dawley Rat Burn Model was used to examine the ability of a liquid pharmaceutical sulfide composition (NaHS) to enhance re-epithelialization in vivo. The experimental design was approved by the Animal Care and Use Committee. All the animals were handled according to the guidelines established by the American Physiology Society and the National Institutes of Health.

Sprague Dawley rats with average body weights of 350 g and average skin surface areas of 435 cm² were caged in an environment in which the temperature and relative humidity were controlled, with alternating 12 hours light/dark cycles, and were given free access to feed and water during acclimation. Ten animals were tested with five controls and five experimental animals.

The animals were anesthetized and intubated using an endotracheal tube. The anesthesia was continued during the course of the experiment. While under anesthesia the backs and flanks of the animals were denuded prior to initiation of the burn model. The anesthetized animals were injected subcutaneously with 1.0 ml of 0.9% saline to prevent deep tissue burn. The Sprague Dawley Rat Burn Model was used with 30% total body surface area (TBSA) full-thickness scald burn under deep anesthesia. The burn area was approximately ˜130 cm². Starting at 48 hours post burn, the animals received daily subcutaneous injections of a liquid pharmaceutical sulfide composition prepared as described in Example 1 (0.53 mg/ml NaHS), at four equally spaced sites in the transition zone between bum eschar and healthy tissue for 14 days.

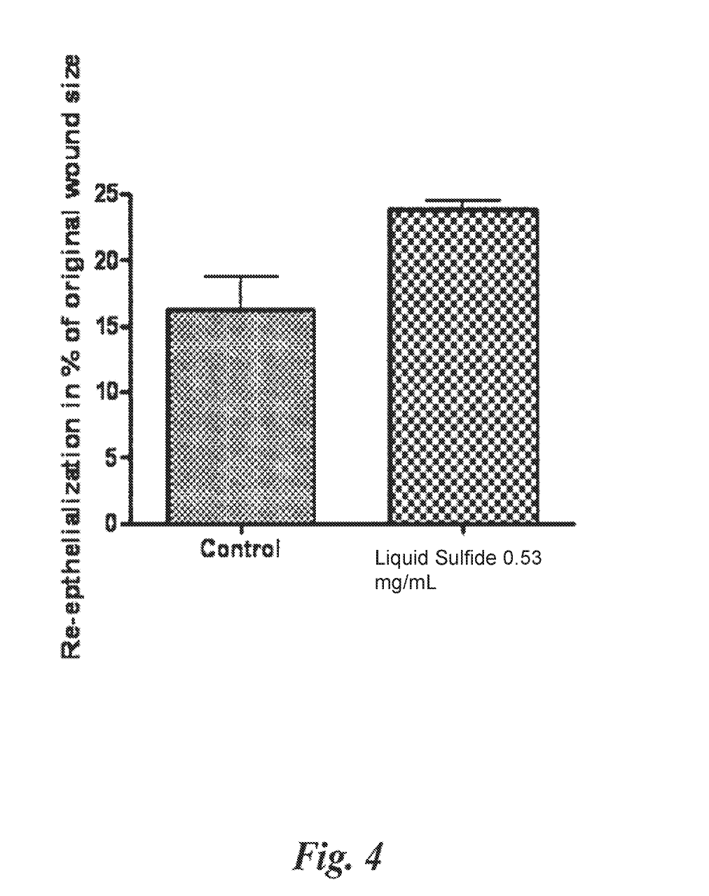

As shown in FIG. 4, re-epithelialization was improved in the presence of the liquid pharmaceutical sulfide composition (0.1 mg NaHS per injection) as compared to the control. Planimetric measurement of the wound surface and re-epithelialization, as well as the ratio of wound contraction, were performed.

### EXAMPLE 9

### A Liquid Sulfide Formulation Stimulates Migration of Endothelial Cells

Cell migration assays were conducted to determine the effect of a liquid formulation of sulfide (NaHS) on endothelial cell migration. HUVECs were serum-starved overnight. Cells were then trypsinized, and 1×10⁵ cells were added to transwells (8 μM pore size) in 100 μl of starvation medium. The test articles, including a liquid formulation of sulfide prepared as described in Example 1 (6 μM or 60 μM NaHS) or vehicle (control), were added to the well containing the transwell inserts at 600 μL volume. Cells were allowed to migrate for 4 hours at 37° C. Non-migrated cells at the top of the transwell filter were removed with a cotton swab. The migrated cells were then fixed in Carson's solution (30 minutes at room temperature) and then stained in toluidine blue (20 minutes at room temperature). Migrated cells were scored in 8 random fields, and the fold-change was determined as compared to the number of migrated cells in control wells.

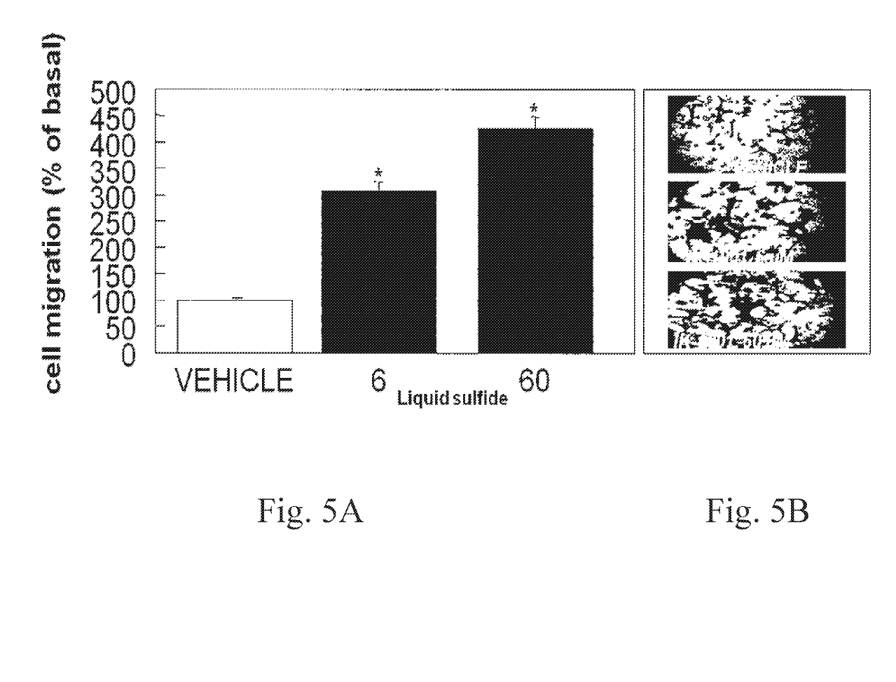

Cells treated with increasing amounts of the liquid formulation of sulfide (NaHS) had greater cell migration in comparison to control cells (FIG. 5A). These results indicate that liquid pharmaceutical sulfide stimulates migration of endothelial cells. Representative photomicrographs of the transwell membrane showing cell migration in vehicle and the liquid formulation of sulfide (NaHS)-treated cells are shown in FIG. 5B.

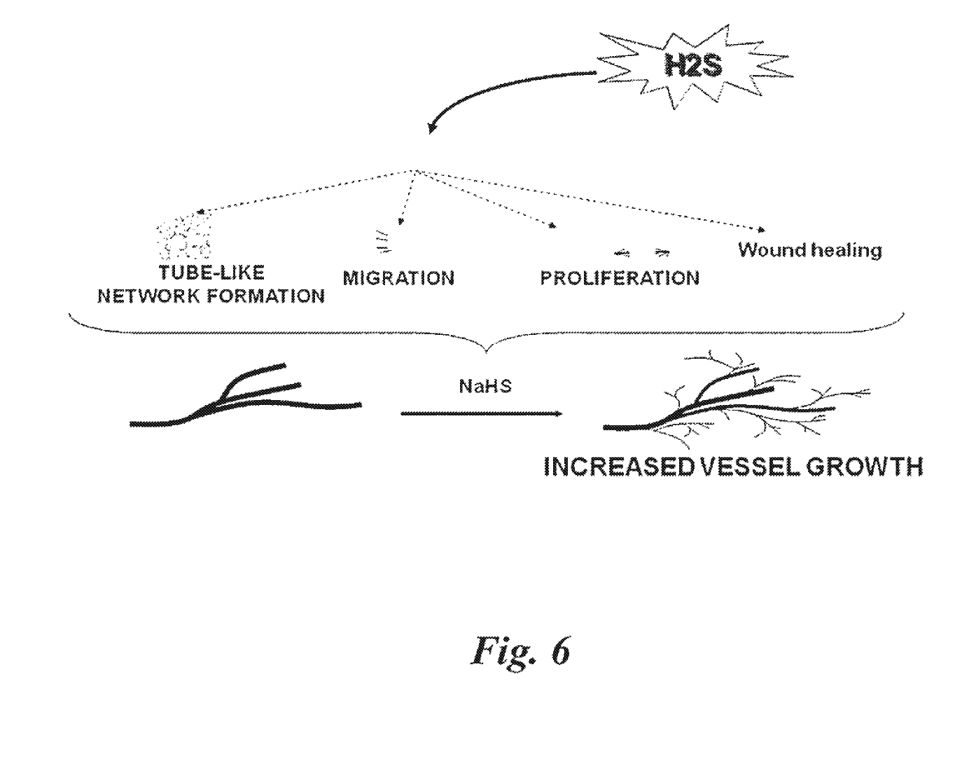

Without wishing to be bound by theory, the diagram depicted in FIG. 6 summarizes various mechanisms, such as endothelial cell migration and proliferation, by which sulfides promote angiogenesis and wound healing.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of stimulating angiogenesis in an animal, tissue or organ, comprising administering to the animal, tissue or organ an effective amount of sulfide.

2. The method of claim 1, wherein said sulfide is administered in a stable liquid pharmaceutical composition comprising said sulfide and a pharmaceutically acceptable carrier, and wherein the concentration, pH, and oxidation products of said sulfide remain within a range of acceptance criteria after storage of said liquid pharmaceutical composition.

3. The method of claim 1, comprising administering to the animal, tissue organ the effective amount of sulfide in combination with an effective amount of nitric oxide.

4-10. (canceled)

11. The method of claim 2, wherein said animal is a mammal or said tissue or organ is a mammalian tissue or organ.

12. (canceled)

13. A method for promoting wound healing in an animal, comprising administering to the animal an effective amount of sulfide, alone or in combination with an effective amount of nitric oxide.

14. The method of claim 13, wherein said sulfide is administered locally, intradermally, intraperitoneally, subcutaneously, or topically.

15. The method of claim 13, wherein said wound healing comprises re-epithelialization of a denuded area of skin of an animal after a burn, trauma, wound, injury, chemotherapy, skin reaction following drug treatment or disease process.

16. (canceled)

17. A method for treating or preventing an injury or disease associated with decreased or insufficient blood flow in an animal, comprising administering to said animal an effective amount of sulfide, alone or in combination with an effective amount of nitric oxide.

18. The method of claim 17, wherein said animal is a mammal.

19-21. (canceled)

22. The method of claim 17, wherein said decreased or insufficient blood flow is cerebral blood flow.

23. The method of claim 17, wherein said decreased or insufficient blood flow is localized within said animal.

24. The method of claim 17, wherein said injury or disease is diabetic foot ulcers.

25. The method of claim 17, wherein said injury or disease is peripheral vascular disease.

26. The method of claim 17, wherein said injury or disease is a coronary injury or disease selected from the group consisting of: congestive heart failure, myocardial ischemia, coronary artery disease, and angina.

27. The method of claim 17, wherein said disease is an ocular disease.

28-29. (canceled)

30. The method of claim 2, wherein the stable liquid pharmaceutical composition is prepared by dissolving one equivalent of hydrogen sulfide gas into one equivalent of sodium hydroxide solution, wherein said composition has a pH in the range of 6.5 to 8.5, wherein said composition has an osmolarity in the range of 250-330 mOsmol/L, wherein said composition has an oxygen content of less than or equal to 5 μM, and wherein said composition comprises oxidation products are the range of 0%-3.0% (w/v) after storage for three months.

31. The method of claim 30, wherein said sulfide is administered intravenously.
